(19) United States
(12) Patent Application Publication
(10) Pub. No.: US 2025/0230413 A1
(43) Pub. Date: Jul. 17, 2025

(54) METHODS AND SYSTEMS FOR THE CULTURE OF CELLS AT LIQUID-LIQUID INTERFACES

(71) Applicant: Queen Mary University of London, London (GB)

(72) Inventors: Julien Ezra Edouard Gautrot, London (GB); Elijah Bryce Alba Mojares, London (GB); Alexandra Chrysanthou, London (GB); Hassan Kanso, London (GB); Minerva Bosch-Fortea, London (GB)

(21) Appl. No.: 18/853,782

(22) PCT Filed: Apr. 5, 2023
(86) PCT No.: PCT/EP2023/058935

§ 371 Date: Oct. 3, 2024

(30) Foreign Application Priority Data
Apr. 6, 2022 (GB) 2205017.3

(51) Int. Cl. C12N 5/077; C12M 1/12

(52) U.S. Cl. CPC C12N 5/0652; C12M 25/02; C12N 2533/30; C12N 2533/50; C12N 2537/10

(57) ABSTRACT

A method of culturing adherent cells at a liquid-liquid interface in a cell culture system. The cell culture system comprises an aqueous cell culture medium and an oil or lipid phase. The oil/lipid phase is functionalised with a conditioning layer that is disposed between the aqueous cell culture medium and the oil/lipid phase. The conditioning layer comprises a self-surface-activating amphiphilic protein or self-surface-activating amphiphilic peptide layer. The method comprises culturing the adherent cells in the cell culture system at the interface between the aqueous cell culture medium and the oil/lipid phase.

### TECHNICAL FIELD

The present disclosure relates to methods of culturing adherent cells, such as adherent stem cells, at a liquid-liquid interface. The disclosure also provides cell culture systems useful in the culture of cells at liquid-liquid interfaces.

### BACKGROUND

Previous studies have shown that adherent cells (e.g. fibroblasts, HaCaT) can be expanded to high density at liquid-liquid interfaces. However, the cell culture systems of the art are not suitable for the long-term proliferation of a broader range of cell types and rely on formulations that are unlikely to be approved by regulatory bodies in the therapeutics and food industry. There remains a need in the art for a method and system for the successful culture of adherent cell types, including primary fibroblasts, HEK293 cells, keratinocytes, MSCs, ESCs and iPSCs, on liquid-liquid interfaces to allow the proliferation and expansion of such cell types without the need for a solid substrate.

Indeed, although liquid-liquid systems have been reported to date, these can involve an array of different chemical species to form stabilised liquid-liquid interfaces suitable for cell adherence and culture. Such chemical species can present deleterious effects on the cell culture method. For example, the use of certain chemical species can contaminate the cell culture and impact regulatory approval testing for a variety of reasons.

It would be desirable to provide a method and system for the culture of cells at liquid-liquid interfaces that is advantageous as compared with existing techniques, and/or to obviate, mitigate and/or ameliorate one or more deficiencies in known methods and system, whether identified herein or otherwise.

### SUMMARY OF THE DISCLOSURE

According to a first aspect, provided herein is a method of culturing adherent cells at a liquid-liquid interface in a cell culture system, the cell culture system comprising:
a. an aqueous cell culture medium; and
b. an oil or lipid phase,
wherein the oil/lipid phase is functionalised with a conditioning layer that is disposed between the aqueous cell culture medium and the oil/lipid phase and the conditioning layer comprises a self-surface-activating amphiphilic protein or self-surface-activating amphiphilic peptide layer;
the method comprising culturing the adherent cells in the cell culture system at the interface between the aqueous cell culture medium and the oil/lipid phase.

According to a second aspect, provided herein is a culture system for the culture of adherent cells at a liquid-liquid interface, the culture system comprising:
a. an aqueous cell culture medium; and
b. an oil or lipid phase, wherein the oil/lipid phase is functionalised with a conditioning layer that is disposed between the aqueous cell culture medium and the oil/lipid phase and the conditioning layer comprises a self-surface-activating amphiphilic protein or self-surface-activating amphiphilic peptide layer;
the system comprising culturing the adherent cells at the interface between the oil/lipid phase and the aqueous cell culture medium.

According to a third aspect, provided herein is a use of the culture system defined in the second aspect for the culture of adherent cells at a liquid-liquid interface.

According to a fourth aspect, provided herein is a method of expanding a population of adherent cells comprising culturing the cells at a liquid-liquid interface according to the first aspect and harvesting the cells from the culture medium.

According to a fifth aspect, provided herein is a bioreactor comprising a culture of adherent cells (optionally stem cells), wherein the adherent cells adhere to a liquid-liquid interface in a cell culture medium defined in the second aspect.

According to a sixth aspect, provided herein is a kit of parts comprising combinations of one or more oils or lipids, and one or more self-surface-activating amphiphilic proteins or self-surface-activating peptides for the culture of adherent cell and optionally stem cells, optionally wherein the oil/lipid(s) and self-surface-activating amphiphilic protein(s)/self-surface-activating amphiphilic peptide(s) are as defined in the first aspect.

### BRIEF DESCRIPTION OF THE FIGURES

FIG. 1. Crosslinked protein nanosheets assembled at liquid-liquid interfaces. (A) Time sweep shows the interfacial storage modulus profile of Novec 7500/PBS interfaces during the assembly of BSA and β-Lactoglobulin (at a final concentration of 1 mg/mL). (B) Frequency sweep at oscillating amplitude of 10-4 rad carried out for the characterization of BSA and β-Lactoglobulin.

FIG. 2. Crosslinked protein nanosheets assembled at liquid-liquid interfaces. Time sweep shows the interfacial storage modulus profile of Novec 7500/PBS interfaces during the assembly of BSA, casein and β-Lactoglobulin (at a final concentration of 1 mg/mL). Washing of excess protein was carried out at time 5000 s.

FIG. 3(I). Growth of MSCs at crosslinked βLG and BSA nanosheets. (A) MSCs growing for 5 days on βLG- and BSA-stabilised interfaces and crosslinked by sulfo-SMCC (1 mg/ml, pH 7.4), followed by coupling of fibronectin. Vinculin staining shows excellent cell adhesion to the interface. Scale bar, 50 μm. (B) Time course showing growth of MSCS on Novec 7500 oil droplets stabilised by BSA nanosheets crosslinked with sulfo-SMCC and functionalised with fibronectin (as above) and on plastic over 22 days. EthD1 is a marker for dead cells. (C) Quantification shows the % of dead cells at different time-points of cells growing on plastic and on corresponding microdroplets. Note that more dead cells are observed on plastic at later timepoints although the percentage of dead cells remains low. (D) Image of the scale expansion of MSCs on crosslinked BSA-stabilised emulsions (cultured in 100 mL conical flasks). Note that cells grow across emulsions and clump together the droplets to form a loose network of cells.

FIG. 3(II). MSC-derived extracellular vesicles secretion from a two-dimensional plastic plate and a liquid substrate (βLG+sulfo-SMCC+RGD). (A) The culture of MSCs after seven days on a 2D and emulsion substrate. (B) Concentration of secreted particles from the 2D plate and emulsion after three, five and seven days in culture. (C) Corresponding concentration over the size profile of EVs secreted from 2D plate and emulsion after seven days.

FIG. 3(III). The EVs secretion and composition isolated from a 2D and emulsion (βLG+sulfo-SMCC+RGD) substrate. (A) Confocal microscopy images of MSCs growth (blue, DAPI; red, phalloidin; green, vinculin; magenta, CD63) on 2D and emulsion after seven days of culture, showing the impact of the substrate stiffness on the cell morphology and phenotype after seven days in culture. (B) Protein content of the isolated EVs from 2D and emulsion substrates on day three, five and seven of culture. Scale bars are 50 μm. Error bars are s.e.m.; n=3.

FIG. 3(IV). Labelling of exosomal RNA isolated from 2D and emulsion (βLG+sulfo-SMCC+RGD). (A) Confocal microscopy images of exosomes captured on magnetic beads labelled with CD63 (red, 555 nm) and RNA (green, 488 nm). (B) Quantification of the labelled exosomal RNA from the corresponding substrates. Scale bars are 50 μm. Error bars are s.e.m.; n=3.

FIG. 4(I). Growth of iPSCs at βLG nanosheets. No PFBC. (A) iPSCs growing for 4 days on tissue culture plastic (TCP) or βLG-fluorinated oil stabilised interfaces (oil: Novec 7500; crosslinked upon assembly/denaturation and after vitronectin adsorption, βLG VN; or via sulfo-SMCC coupling at 1 mg/mL, followed by vitronectin adsorption and coupling, βLG SMCC VN). Oct 3/4 and Nanog staining shows pluripotency is maintained. Scale bar is 50 μm. (B) Histogram showing distribution of mean fluorescence intensity of Oct 3/4 and Nanog of iPSCs cultured on TCP and βLG-fluorinated oil stabilised interfaces. (C) Table summarising the percentage of iPSCs positive for both pluripotency markers (n=3). (D) Sample micrographs of iPSC clusters grown on TCP and βLG-fluorinated oil stabilised interfaces after seeding and after 48 hours. Scale bar is 100 μm. (E) Quantification shows area of clusters measured at T=0 and T=48 hours. Relative growth of clusters is measured as the ratio of areas at T=48 hours and T=0 hour (n=3). (F) iPSCs growing for 4 days on glass and βLG-SMCC-VN/Fluorinated oil microdroplets with different microdroplet size. Cells grown on the microdroplets are cultured either in static conditions or in dynamic conditions via gyro-rocker agitation. Staining shows positive pluripotency markers Oct 3/4 and Nanog. (G) Flow cytometry of iPSCs grown on TCP and βLG-SMCC-VN/Fluorinated oil microdroplets of different sizes and cultured either in static conditions or in dynamic conditions. Dot plots show iPSCs grown in different conditions are positive for intracellular markers for pluripotency, Oct 3/4 and Nanog, as well as surface markers for pluripotency, SSEA-4 and TRA-1-60-R.

FIG. 4(II). Cardiomyocyte differentiation on 100 μm βLG-SMCC-VN microdroplets (bLG (1 mg/mL), crosslinked with SMCC and further coupled with vitronectin) after glucose starvation. (A) Maximum projection of confocal images. iPSCs (40,000 cells) were seeded on microdroplets in a 24-well plate and maintained in iPSC medium for 4 days before differentiation was performed. CHIR99021 concentration used was 4 μM. (B) Striated pattern of TNNT2 and α-actinin can be observed as indicated by white arrows. Scale bar is 50 μm.

FIG. 4(III). Percentage of TNNT2+ when differentiation is initiated 2 days after seeding on microdroplets (functionalised with bLG (1 mg/mL), crosslinked with SMCC and further coupled with vitronectin) or EB formation (Trial 2). Cells were cultured in CDM3 medium with varying CHIR and WntC59 concentrations. Cells were gated with FSC-A×SSC-A. Doublets were removed from analysis with FSC-A×FSC-W. Dead cells were excluded from analysis with Zombie Aqua viability stain. CHIR99021 6 μM consistently works across conditions except for 230 μm microdroplets when 5 μM of Wnt-C59 is used. Higher CHIR99021 concentration only works for microdroplets and only when followed by 5 μM Wnt-C59.

FIG. 4(IV). Representative maximum projection of confocal images of iPSC-CMs generated after 7 days of differentiation on microdroplets functionalised with bLG (1 mg/mL), crosslinked with SMCC and further coupled with vitronectin. Cells were cultured in CDM3 medium with varying CHIR and WntC59 concentrations. Dosing regimen for each CHIR99021/Wnt-C59 (μM) is 8/5, 6/5, and 6/5 for cultured on 150 μm, 230 μm, and as EB. Different morphologies can be observed. Rounded cells can be seen in the upper right portion of the leftmost panel. Scale bar is 50 μm.

FIG. 4(V). as Circular morphology of TNNT2+ cardiomyocytes from EBs with the differentiation regimen of CHIR99021 6 μM and Wnt-C59 5 μM. The morphology is very different from the previous figure as most of the cells are rounded and the sarcomeres are not outstretched yet. Scale bar is 50 μm.

FIG. 5. Growth of HEK293T at BSA nanosheets crosslinked with sulfo-SMCC (1 mg/mL) and functionalised with fibronectin. (A) Images show HEK293T cells growing for 5 days on BSA-stabilised microdroplets (Novec 7500). Scale bar, 100 μm. Quantification shows the growth of HEK293T cells on plastic and on BSA interface, examined via CCK-8 cell viability assays. (B) HEK293T cells growing for 5 days on BSA-stabilised interfaces. Ki67 stains proliferating cells Scale bar, 100 μm. Quantification shows % of proliferating cells at different time-points of cells growing on BSA interfaces. (C) Time course showing growth of HEK293T on BSA interfaces and on plastic over 22 days. EthD1 stains dead cells. (D) Quantification shows % of dead cells at different time-points of cells growing on plastic and on BSA interfaces. (E) HEK293T growing on BSA interfaces for 22 days showing high proliferation after long-term culture. Scale bar, 100 μm.

FIG. 6(I). Growth of MSCs at crosslinked fibrinogen nanosheets. (A) Evolution of interfacial moduli of interfaces (Novec 7500-PBS) upon formation of fibrinogen nanosheets (fibrinogen concentration 1 mg/mL), followed by washing of the aqueous phase to remove excess fibrinogen and further crosslinking via thrombin treatment (concentrations indicated). (B) MSCs growing for 7 days on resulting fibrinogen interfaces with varying levels of thrombin crosslinking, and fibronectin adsorption (from 20 μg/mL solutions). (C) MSCs growing for 7 days within fibrinogen-stabilised Novec 7500 emulsions, with different degrees of thrombin crosslinking and fibronectin adsorption (as above). Hoechst staining.

FIG. 6(II). MSCs growing for 5 days on fibrinogen-stabilised pinned droplet interfaces with varying levels of thrombin crosslinking, and fibronectin adsorption (from 20 g/mL solutions). Actin (red), DAPI (blue) and Fibrinogen-488 (green) staining.

FIG. 6(III). MSCs growing for 7 days within fibrinogen-stabilised Novec 7500 emulsions, with different degrees of thrombin crosslinking and fibronectin adsorption (from 20 μg/mL solutions). Actin (red), DAPI (blue) and Fibronectin (green) staining.

FIG. 6(IV). Toughness of interfaces (Novec 7500-PBS) upon formation of fibrinogen nanosheets (fibrinogen concentration 1 mg/mL), followed by washing of the aqueous phase to remove excess fibrinogen and further crosslinking via thrombin treatment (concentrations indicated).

FIG. 7. (A) Time sweep shows the interfacial storage modulus profile of Novec 7500/PBS interfaces during the assembly of native, NB, AC and MA BSA (at a final concentration of 1 mg/mL). (B) Frequency sweep carried out for the characterization of native, NB, AC and MA BSA.

FIG. 8. Crosslinking of functionalised BSA nanosheets. MA-BSA (A) and AC-BSA (B) assembly at liquid-liquid interfaces (Novec 7500; 1 mg/mL protein concentration), followed by increase in pH to 10.5 to induce crosslinking of nanosheets, monitored via interfacial rheology.

FIG. 9. Images of peptide-functionalised droplets. NB-BSA emulsions (left) stained with SAMSA fluorescein to assess the presence of maleimide moieties. Ac-BSA (centre) and MA-BSA (right) functionalised with fluorescent RGD peptides. Scale bar, 100 μm.

FIG. 10. Images of cells growing on corresponding microdroplets. (A) MSCs, fibroblasts and HEK293T cells growing on reactive BSA nanosheets in fluorinated oil droplets for 5 days. Scale bar, 100 μm (B) Fibroblasts and HEK293T cells growing on reactive BSA nanosheets in rapeseed oil droplets for 5 days. Scale bar, 100 μm.

FIG. 11. Supercharged protein nanosheets assembled at liquid-liquid interfaces. (A) Time sweep shows the interfacial storage modulus profile of Novec 7500/PBS interfaces during the assembly of native, cationic and anionic BSA (at a final concentration of 1 mg/mL). (B) Frequency sweep at oscillating amplitude of 10⁻⁴ rad carried out for the characterization of native, cationic and anionic BSA.

FIG. 12. (A) Epifluorescence microscopy images were with rabbit (green, AB F3648) and mouse (red, AB ab90395) antibodies and species specific secondary antibodies conjugated. (B) Representative surface plasmon resonance data showing binding of the proteins to the SPR-Au treated with perflurodecanethiol chip showing the level of binding for each protein. (C) Bar graph of the response unit for native, anionic and cationic BSA. (D) Investigation of the FN or collagen binding at the pre-adsorbed protein layer on the SPR chip. (E) Shows the comparison of the FN or collagen on the native BSA and the charge modified BSA. Errors bars are s.e.m; n=3.

FIG. 13. Human primary keratinocytes spreading at liquid-liquid interfaces after seven days of culture at TCP, native BSA-fibronectin and cationic BSA-fibronectin. Images are corresponding nuclear stainings (White, Hoechst 33342).

FIG. 14. Formation and mechanical properties of BSA, NB-BSA, AC-BSA and MA-BSA nanosheets (absorbed at 1 mg/mL in PBS) at oil (Novec 7500)-water interfaces. Evolution of interfacial storage moduli during the adsorption of albumins, including crosslinking with sulfo-SMCC (2 mg/mL) at 6000 s at oil-PBS interface.

FIG. 15. Growth of MSCs on fluorinated oil/modified BSA emulsions functionalised with RGD and crosslinked with sulpho-SMCC. (A) The stability of fluorinated oil bioemulsions was evaluated over time by measuring droplet volume distribution using Mastersizer2000. Graphs show particle size distribution at day 0, day 7 and day 15 of NB-BSA, Ac-BSA and MA-BSA bioemulsions. Values are mean for average radii+SD from triplicate measurements. (B) Bioemulsions formed after Michael addition reaction to conjugate RGD-FITC to BSA and NB-BSA. Scale bar, 50 μm. Graph shows relative mean fluorescence intensity of RGD-FITC at the surface BSA and NB-BSA bioemulsions, characterised by epifluorescence microscopy (with respect to interfaces formed on BSA). Values are mean±SD (n>20 droplets/condition; ***, P<0.001). (C) Bioemulsions formed after radical thiol-ene coupling of RGD-FITC to BSA, AC-BSA and MA-BSA. Scale bar, 50 μm. Graph shows relative mean fluorescence intensity of RGD-FITC at the surface of BSA, AC_BSA and MA-BSA bioemulsions, characterised by epifluorescence microscopy (with respect to interfaces formed on BSA). Values are mean±SD (n>20 droplets/condition; ***, P<0.001). (D) Immunofluorescence images of FAs (Vinculin, green) of cells growing on the modified-BSA bioemulsions for 5 days. Magnifications show mature FAs on crosslinked interfaces. Scale bar, 50. (E) Matrix deposition (fibronectin, green) on modified-BSA bioemulsions after 7 days of culture. Scale bar, 100 μm. (F) Graph shows MSCs expansion after 7 days of culture in 2D and at the surface of crosslinked and non-crosslinked NB-BSA, AC-BSA and MA-BSA bioemulsions, characterised by CyQuant (with respect to number of cells at day 1). Values are mean±SD of from 3 different experiments (NS, P>0.05; *, P<0.05; **, P<0.01). (G) Graph shows the percentage of the MSCs growing after one day in culture at the surface of crosslinked and non-crosslinked NB-BSA, AC-BSA and MA-BSA bioemulsions, characterised by CyQuant (with respect to initial number of cells seeded). Values are mean±SD of from 3 different experiments (NS, P>0.05; **, P<0.01).

FIG. 16. Growth of MSCs on rapeseed oil/modified BSA emulsions functionalised with RGD and crosslinked with sulpho-SMCC. (A) Timelapse images show MSCs growing on rapeseed oil bioemulsions with NB-BSA, AC-BSA, and MA-BSA crosslinked with SH-SMCC. Scale bar, 200 μm. Graphs show the number of cells on 2D and the different bioemulsions at different timepoints. Growth dynamics of MSCs was measured using CyQuant. Values are mean±SD from 3 different experiments (*, P<0.05; ***, P<0.001; ****, P<0.0001). (B) Graph show the percentage of the initial number of cells that attach to both on 2D and to the rapeseed oil bioemulsions after seeding. Values are mean±SD from 3 different experiments (**, P<0.01; ***, P<0.001). (C) Graph show cell expansion on 2D and on rapeseed bioemulsions after 7 days in culture. Values are mean±SD from 3 different experiments (*, P<0.05). (D) MSCs growing on 2D for 2 days after harvesting from 7 days MA-BSA bioemulsions. Re-seeding was done by placing the bioemulsions on 2D (upper) or after pre-treatment with tryspsin (lower). Scale bar, 50 μm.

FIG. 17. Growth of MSCs on fluorinated oil/MA-BSA emulsions functionalised with RGD and crosslinked with sulpho-SMCC. Scale-up format. (A) Bioreactor format. 250 ml Erlenmeyer flask containing 10 ml of bioemulsions and 50 ml of medium. (B) Graph show the percentage of the initial number of cells that attached to the substrate on 2D and to the bioemulsions 1 day after seeding. Values are mean±SD from 3 different experiments (*, P<0.05). (C) MSCs growing on MS-BSA bioemulsions for 15 days in a 250 ml flask under agitation. Note cells cover all the available surface of the bioemulsions. Scale bar, 200 μm. (D) Live/Dead staining (left) of MSCs cells growing on 2D and on MA-BSA crosslinked bioemulsions for 1, 7 and 15 days. Scale bar, 100 μm. Quantification (right) shows the percentage of live cells at 1, 7 and 15 days on 2D and on the bioemulsions. Values are mean±SD from 3 different experiments (NS, P>0.05). (E) Gating strategy (left) and CD73, CD105 and CD90 expression percentages (right) of MSCs growing on 2D and on bioemulsions recovered at day 15. Values are mean±SD from 3 different experiments (NS, P>0.05). Cells growing on 2D (top panels) or fibrin gels (bottom panels) for 5 days after recovery from 15-day bioemulsions. Harvesting and re-seeding was done using different methods. Scale bar, 100 μm.

### DEFINITIONS

It will be appreciated that a surfactant (surface active agent) may be understood to be a substance which lowers the surface tension of the phase (e.g. aqueous cell culture medium or oil/lipid phase) in which it is provided, and/or the interfacial tension with other phases (see, for example, IUPAC Gold Book 3.0.1).

A “chemical surfactant” as defined herein may be understood to refer to a non-proteinaceous/peptidic surfactant. Various characteristics of such chemical surfactants are provided herein, such as maximum molecular weights, specific chemical species, etc. and the skilled person will well-understand the types of surfactants that are envisaged.

As used herein, the terms “self-surface-activating amphiphilic protein” and/or “self-surface-activating amphiphilic peptide” are intended to refer to the fact that the amphiphilic protein and/or amphiphilic peptide enable formation of a surface activated conditioning layer by themselves. In other words, additional surfactant (e.g. chemical surfactant) is not needed to form the conditioning layer.

The term “conditioning layer” may be understood as a complete layer separating one phase from another (e.g. separating the aqueous cell culture medium and oil/lipid phase). A conditioning layer can take many forms, such as in the production of an emulsion and/or a sheet (e.g. a planar sheet).

The terms “about” or “around” generally encompass or refer to a range of values that one skilled in the art would consider equivalent to the recited values (e.g. having substantially the same function or result, and/or achieved in substantially the same way). Suitably, where such a term is used in relation to a numerical value, it can represent (in increasing order of preference) a 10%, 5%, 2%, 1% or 0% deviation from that value.

The term “consists essentially of” is used herein to denote that a given method, use, product or component thereof consists of only designated materials and optionally other materials that do not materially affect the characteristic(s) of the method, use, product or component thereof. In the context of a conditioning layer, for example, this term may be understood to denote that the conditioning layer consists of only the designated “self-surface-activating amphiphilic protein and/or self-surface-activating amphiphilic peptide” and optional other materials which do not affect the surfactant characteristics of that conditioning layer. Similar considerations apply to given products and uses. Suitably, a method, use, product or component thereof which consists essentially of a designated material (or materials) comprises greater than or equal to about 60% of the designated material(s), more suitably greater than or equal to about 80%, more suitably greater than or equal to about 90%, more suitably greater than or equal to about 95%, more suitably greater than or equal to about 99% of the designated material(s), on a weight basis. Higher levels are preferred.

The term “consists of” is used herein to denote that a given method, use or product consists of only designated materials only (optionally with de minimis additional materials: those too small to be meaningful or taken into consideration, immaterial). In the context of a conditioning layer, for example, this term may be understood to denote that the conditioning layer consists of only the designated “self-surface-activating amphiphilic protein and/or self-surface-activating amphiphilic peptide”. Similar considerations apply to given products and uses. Suitably, a method, use or product which consists of a designated material (or materials) comprises greater than or equal to about 60% of the designated material(s), more suitably greater than or equal to about 80%, more suitably greater than or equal to about 90%, more suitably greater than or equal to about 95%, such as about 99% of the designated material(s), on a weight basis. Higher levels are preferred.

The term “substantially” as used herein is intended to modify a quality such that a given feature need not be “exactly” in accordance with that quality. Suitably, this modifier may indicate a deviation from the quality given of up to about 10%, such as up to about 5%, such up to about 4%, such as up to about 3%, such as up to about 2%, such as up to about 1%, such as about 0%. Generally speaking, lower deviation may be preferred. By way of example, if something substantially does not comprise a substance, this may be interpreted as also referring to a material having up to about 5% of the substance, for example. Any amounts here are wt % amounts.

As used herein, a molecular weight (e.g. expressed in Daltons, Da) may be understood to refer to a number-average/weight average molecular weight in the case of polymers (as specified) or the exact molar mass of a protein. Molecular weight may suitably be measured in accordance by gel permeation chromatography and molar masses via mass spectrometry (e.g. MALDI-TOF) or gel electrophoresis (approximate).

When a plurality of substances is noted as being “at a concentration” (or other amount/quantity), this is to be understood to refer to the total concentration (or other amount/quantity) of the collection of substances making up the plurality. For example, if “the surface-activating amphiphilic protein and/or self-surface-activating amphiphilic peptide are provided at a concentration of at least . . . ” and there are both surface-activating amphiphilic proteins and surface-activating amphiphilic peptides, then the concentration refers to the total concentration of the collection of those proteins and peptides in the aqueous cell culture medium.

### DETAILED DESCRIPTION

### Cell Culture Systems

According to a first aspect, provided herein is a method of culturing adherent cells at a liquid-liquid interface in a cell culture system, the cell culture system comprising:
a. an aqueous cell culture medium; and
b. an oil or lipid phase,
wherein the oil/lipid phase is functionalised with a conditioning layer that is disposed between the aqueous cell culture medium and the oil/lipid phase and the conditioning layer comprises a self-surface-activating amphiphilic protein or self-surface-activating amphiphilic peptide layer; the method comprising culturing the adherent cells in the cell culture system at the interface between the aqueous cell culture medium and the oil/lipid phase.

The methods of the present disclosure employ a novel cell culture system that provides optimal conditions for the culture of adherent cells at a liquid-liquid interface. The cell culture systems are particularly suited to the culture of adherent cells and stem cells, although any adherent cell types (e.g. progenitor cells or other cell lines) can be used. Cell populations cultured according to methods of the disclosure grow just as well, and in some cases better, compared to traditional cell culture systems that use a solid substrate such as plastic.

Moreover, the present disclosure is based on the surprising finding that careful selection of the protein/peptide making up the conditioning layer can improve existing systems and reduce the need for chemical species, such as chemical surfactants and/or additional polymeric layers. Specifically, the use of a self-surface-activating amphiphilic protein or self-surface-activating amphiphilic peptide in the conditioning layer enables that protein/peptide to form essentially the entire conditioning layer alone, without needing those additional chemical components. Accordingly, in some implementations, the conditioning layer and oil/lipid phase substantially do not comprise a chemical surfactant. Various provisos are provided herein to highlight numerous other chemical species which can be omitted with the advantageous method of the present disclosure. Overall, the ability to omit these species streamlines existing technologies. Moreover, such chemical species might otherwise be deleterious on the cell culture method for a variety of other reasons (e.g. those which may cause cell culture methods to fail regulatory approval testing) and so the ability to omit these presents yet further advantages.

For example, as discussed below, the emulsion-based cell culture systems may permit direct administration of the emulsion containing cultured cells directly to a patient or to a tissue engineering platform (e.g. for a therapeutic use thereof). It will be appreciated that chemical species, such as chemical surfactants, could mean that such emulsions are not suitable for such implementations. Omitting these, by careful selection of the self-surface-activating amphiphilic protein or self-surface-activating amphiphilic peptide, thereby obviates/mitigates this issue.

The cell culture systems comprise an aqueous cell culture medium and an oil/lipid phase. The aqueous cell culture medium may be provided by an aqueous buffer, but the conditioning layer may be assembled in deionised water, an aqueous buffer or cell culture medium, before exchange of the aqueous phase to cell culture medium or a suitable buffer. The oil/lipid phase comprises a conditioning layer that is assembled between the aqueous cell culture medium and the oil/lipid phase. The conditioning layer comprises a self-surface-activating amphiphilic protein or self-surface-activating amphiphilic peptide layer.

The conditioning layer functionalises the oil/lipid phase to allow the efficient and longer-term culture of adherent cells. When cells are cultured, they grow on the surface of the functionalised oil/lipid (i.e. on the conditioning layer). Unlike in cell cultures systems of the art, the cell culture systems of the disclosure allow the culture of the cells at the liquid-liquid interface without causing disruption of the surface of the oil/lipid, by providing optimum elasticity (as measured by interfacial stress-relaxation experiments). Indeed, the inventors have surprisingly found that elastic interfaces, despite their ultra-low dimension (a few nm to a few tens of nm) can can display sufficient elasticity to resist cell-mediated or shear mediated deformations, rather than being dissipated through fracture.

Contrary to the wisdom in the art, the inventors found that a high modulus is not required for the culture of the cells at the liquid-liquid interface, and indeed would be detrimental to the culture of cells given such a higher modulus can make the interface brittle. The cells may adhere via integrin-mediated adhesion and cytoskeleton assembly. The conditioning layer is provided such that it has the suitable mechanical properties discussed herein to enable to long-term culture of adherent cells, including stem cells.

The present culture systems and methods allow the culture of adherent cells in a scalable system that can easily be used to provide a large number of cultured cells due to the increased proliferation rate of cells cultured using the system, and the increased surface area of systems that are in the form of an emulsion. The cell systems presented may also allow the production of a large amount of proteins or other molecules synthesised by the cells, for example the production of antibodies or recombinant proteins and growth factors by cells. The cell culture systems may also allow the longer-term culture of cells such as MSCs and HPKs than seen with the cell culture systems of the art.

### Provisos

The method may carry the proviso that the conditioning layer and/or oil/lipid phase (optionally the conditioning layer, optionally the conditioning layer and oil/lipid phase) substantially do not comprise a chemical surfactant, particularly the chemical surfactants discussed below. In preferred implementations throughout, the conditioning layer and oil/lipid phase substantially do not comprise a chemical surfactant, particularly the chemical surfactants discussed below.

The method may carry the proviso that the conditioning layer and/or oil/lipid phase (optionally the conditioning layer, optionally the conditioning layer and oil/lipid phase) substantially do not comprise a surfactant selected from the group consisting of:
pentafluorobenzoyl chloride (PFBC), pentadecafluorooctanoyl chloride (PDFC), octanoyl chloride, sebacoyl chloride and/or heptadecanoyl chloride.

In some implementations, the method may carry the proviso that the conditioning layer and/or oil/lipid phase (optionally the conditioning layer, optionally the conditioning layer and oil/lipid phase) substantially do not comprise an acyl chloride surfactant.

The method may carry the proviso that the conditioning layer and/or oil/lipid phase (optionally the conditioning layer, optionally the conditioning layer and oil/lipid phase) substantially do not comprise a reactive surfactant (e.g. able to form covalent bonds with the self-surface-activating amphiphilic protein or self-surface-activating amphiphilic peptide of the conditioning layer). In some implementations, the method may carry the proviso that the conditioning layer and/or oil/lipid phase (optionally the conditioning layer, optionally the conditioning layer and oil/lipid phase) substantially do not comprise a reactive surfactant having one or more reactive functional groups selected from acyl chlorides, isocyanates, maleimides, succinimide esters, azides, alkynes, alkyl halides or thiols.

The method may carry the proviso that the conditioning layer and/or oil/lipid phase (optionally the conditioning layer, optionally the conditioning layer and oil/lipid phase) substantially do not comprise a small molecule chemical surfactant, such as a chemical surfactant having a molecular weight up to about 500 Da, optionally up to about 600 Da, optionally up to about 700 Da, optionally up to about 800 Da, optionally up to about 900 Da, optionally up to about 1,000 Da.

The method may carry the proviso that there is no polymer, protein or peptide layer between the conditioning layer and the oil/lipid phase.

### Self-Surface-Activating Amphiphilic-Protein/Peptide Component of the Conditioning Layer

The protein layer provides the support for culturing the cells at the liquid-liquid interface. The protein/peptide layer facilitates the adherence of the adherent cells.

The self-surface-activating amphiphilic-protein and/or self-surface-activating amphiphilic-peptide may have a molecular weight of:
at least about 500 Da, optionally at least about 2500 Da, optionally at least about 5,000 Da, optionally at least about 7,500 Da, optionally at least about 10,000 Da, optionally at least about 20,000 Da; and/or
at most about 10 MDa, optionally at most about 7.5 MDa, optionally at most about 5 MDa; and/or
about 5 to 500 kDa, optionally about 10 to 350 kDa, optionally about 20 to 350 kDa.

The conditioning layer may consist essentially of the self-surface-activating amphiphilic protein or self-surface-activating amphiphilic peptide. In some implementations, the conditioning layer consists of the self-surface-activating amphiphilic protein or self-surface-activating amphiphilic peptide. For example, the method may further comprise washing to remove other, non-proteinaceous, components.

The self-surface-activating amphiphilic protein or self-surface-activating amphiphilic peptide may be crosslinked after assembly at the oil/lipid-water interface.

The self-surface-activating amphiphilic protein or self-surface-activating amphiphilic peptide may crosslink spontaneously at the oil/lipid-water interface.

The self-surface-activating amphiphilic protein or self-surface-activating amphiphilic peptide may be fibrinogen. Additionally, the method may further comprise incubation in a crosslinking protease such as thrombin after assembly of the self-surface-activating amphiphilic protein or self-surface-activating amphiphilic peptide at the liquid-liquid interface.

The self-surface-activating amphiphilic protein or self-surface-activating amphiphilic peptide may be modified with charged moieties (e.g. the protein/peptide is anionic or cationic such as anionic BSA, optionally anionic and in combination with a collagen layer between the conditioning layer and the aqueous cell culture medium, optionally with anionic BSA).

The self-surface-activating amphiphilic protein or self-surface-activating amphiphilic peptide may be modified with an agent selected from N-hydroxysuccinimide esters, pentafluorobenzoic esters, anhydrides, maleimides, acrylates, methacrylates, thiols, alkynes, azides, cyclic alkynes, haloalkanoates, isocyanates and/or epoxides.

The self-surface-activating amphiphilic protein or self-surface-activating amphiphilic peptide may be modified with an agent selected from N-hydroxysuccinimide esters, pentafluorobenzoic esters, maleimides, acrylates, methacrylates, thiols, alkynes, azides, cyclic alkynes, haloalkanoates, isocyanates and/or epoxides.

The self-surface-activating amphiphilic protein or self-surface-activating amphiphilic peptide may be modified with an agent selected from:
1. Sulfo-SMCC (4-(N-Maleimidomethyl)cyclohexane-1-carboxylic acid 3-sulfo-N-hydroxysuccinimide ester sodium salt);
2. Succinimidyl-trans-4-(N-maleimidylmethyl)cyclohexane-1-carboxylate;
3. 3-(Maleimido)propionic acid N-hydroxysuccinimide ester;
4. 6-Maleimidohexanoic acid N-hydroxysuccinimide ester;
5. Poly(ethylene glycol) (N-hydroxysuccinimide 5-pentanoate) ether N′-(3-maleimidopropionyl)aminoethane;
6. Poly(ethylene glycol) bis (N-hydroxysuccinimide) ester;
7. Poly(ethylene glycol) dithiol;
8. Poly(ethylene glycol) bismaleimide;
9. Poly(ethylene glycol) bisacrylate;
10. Poly(ethylene glycol) bismethacrylate;
11. Poly(ethylene glycol) bisazide;
12. Tetra arm and multi-arm polyethylene glycol, end functionalised with thiol, succinimide (e.g. succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester),
maleimide (e.g. PEG-maleimide, PEG-bis maleimide, PEG-tetra arm maleimide or other maleimide-functionalised polymers),
acrylate (e.g. Ac-BSA),
methacrylate (e.g. MA-BSA),
norbornene (e.g. NB-BSA),
vinyl/olefin,
tyramine,
tyrosine,
reactive (cross-linkable) aromatic,
alkyne,
strained alkyne,
azide, or
haloalkanoate;
preferably wherein the self-surface-activating amphiphilic protein or self-surface-activating amphiphilic peptide is modified with an agent selected from acrylate, methacrylates and norbornene;
13. Thiolated hyaluronic acid;
14. Thiolated gelatin;
15. Thiolated dextran;
16. Thiolated polymers;
17. Anhydrides, such as of palmitic acid and adipic acid, and/or mixtures of aliphatic acids and acetic anhydride (e.g. including palmitic acid, adipic acid and/or acetic anhydride); and/or
18. Mixtures of aliphatic carboxylic acids and acetic anhydride.

The self-surface-activating amphiphilic protein or self-surface-activating amphiphilic peptide may be modified with an agent selected from:
1. Sulfo-SMCC (4-(N-Maleimidomethyl)cyclohexane-1-carboxylic acid 3-sulfo-N-hydroxysuccinimide ester sodium salt);
2. Succinimidyl-trans-4-(N-maleimidylmethyl)cyclohexane-1-carboxylate;
3. 3-(Maleimido)propionic acid N-hydroxysuccinimide ester;
4. 6-Maleimidohexanoic acid N-hydroxysuccinimide ester;
5. Poly(ethylene glycol) (N-hydroxysuccinimide 5-pentanoate) ether N′-(3-maleimidopropionyl)aminoethane;
6. Poly(ethylene glycol) bis (N-hydroxysuccinimide) ester;
7. Poly(ethylene glycol) dithiol;
8. Poly(ethylene glycol) bismaleimide;
9. Poly(ethylene glycol) bisacrylate;
10. Poly(ethylene glycol) bismethacrylate;
11. Poly(ethylene glycol) bisazide;
12. Tetra arm and multi-arm polyethylene glycol, end functionalised with thiol, succinimide (e.g. succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester),
   maleimide (e.g. PEG-maleimide, PEG-bis maleimide, PEG-tetra arm maleimide or other maleimide-functionalised polymers),
   acrylate (e.g. Ac-BSA),
   methacrylate (e.g. MA-BSA),
   norbornene (e.g. NB-BSA),
   vinyl/olefin,
   tyramine,
   tyrosine,
   reactive (cross-linkable) aromatic,
   alkyne,
   strained alkyne,
   azide, or
   haloalkanoate;
   preferably wherein the self-surface-activating amphiphilic protein or self-surface-activating amphiphilic peptide is modified with an agent selected from acrylate, methacrylates and norbornene;
13. Thiolated hyaluronic acid;
14. Thiolated gelatin;
15. Thiolated dextran; and/or
16. Thiolated polymers.

The self-surface-activating amphiphilic protein or self-surface-activating amphiphilic peptide may be functionalised prior to modification with the agent to enable reaction between the agent and modified functionality (e.g. for 7, 13, 14, 15 and/or 16 above). In some implementations, the self-surface-activating amphiphilic protein or self-surface-activating amphiphilic peptide are functionalisable. The self-surface-activating amphiphilic protein or self-surface-activating amphiphilic peptide may be a functionalisable albumin.

The self-surface-activating amphiphilic protein or self-surface-activating amphiphilic peptide may be crosslinked after assembly at the oil/lipid-water interface by protein/peptide denaturation. Suitably, this is achieved at neutral pH, or by pH shift, optionally a shift towards alkaline, such as to around pH 10 to 11, such as about pH 10.5.

The self-surface-activating amphiphilic protein or self-surface-activating amphiphilic peptide may be provided with one or more crosslinking moieties (optionally two, three, four or more crosslinking moieties, such as oligofunctional or polyfunctional).

The one or more crosslinking moieties may be selected from:
thiol,
succinimide (e.g. succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester),
maleimide (e.g. PEG-maleimide, PEG-bis maleimide, PEG-tetra arm maleimide or other
maleimide-functionalised polymers),
acrylate (e.g. Ac-BSA),
methacrylate (e.g. MA-BSA),
norbornene (e.g. NB-BSA),
vinyl/olefin,
tyramine,
tyrosine,
reactive (cross-linkable) aromatic,
alkyne,
strained alkyne,
azide,
haloalkanoate; or
anhydride (e.g. the including palmitic acid and adipic acid, or mixtures of aliphatic carboxylic acid, including palmitic acid, adipic acid and/or acetic anhydride).

The one or more crosslinking moieties may be selected from:
thiol,
succinimide (e.g. succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester),
maleimide (e.g. PEG-maleimide, PEG-bis maleimide, PEG-tetra arm maleimide or other maleimide-functionalised polymers),
acrylate (e.g. Ac-BSA),
methacrylate (e.g. MA-BSA),
norbornene (e.g. NB-BSA),
vinyl/olefin,
tyramine,
tyrosine,
reactive (cross-linkable) aromatic,
alkyne,
strained alkyne,
azide, or
haloalkanoate.

In preferred implementations, the one or more crosslinking moieties are selected from acrylate, methacrylates, norbornene and anhydrides.

In preferred implementations, the one or more crosslinking moieties are selected from acrylate, methacrylates and norbornene.

In preferred implementations, the one or more crosslinking moieties are selected from anhydrides or mixtures of aliphatic carboxylic acids and acetic anhydride.

The self-surface-activating amphiphilic protein or self-surface-activating amphiphilic peptide may be provided with two, three, four or more crosslinking moieties, such as oligofunctional or polyfunctional.

The self-surface-activating amphiphilic protein or self-surface-activating amphiphilic peptide may be crosslinked after assembly at the oil/lipid-water interface with a crosslinker (optionally a crosslinker having two, three, four or more crosslinking moieties, such as an oligofunctional crosslinker or a polyfunctional crosslinker).

In some implementations, the self-surface-activating amphiphilic protein or self-surface-activating amphiphilic peptide is crosslinked with:
a thiolated compound (such as a thiolated polymer or oligomer, e.g. thiolated hyaluronic acid, thiolated carboxymethyl cellulose, thiolated dextran and/or thiolated gelatin),
a succinimide (e.g. succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester),
a maleimide (e.g. PEG-maleimide, PEG-bis maleimide, PEG-tetra arm maleimide or other maleimide-functionalised polymers),
an acrylate (e.g. with BSA),
a methacrylate (e.g. with BSA),
a norbornene (e.g. with BSA),
a vinyl compound/olefin,
tyramine,
tyrosine,
a reactive aromatic compound,
alkyne,
strained alkyne,
azide, or
haloalkanoate.

In preferred implementations, the one or more crosslinking moieties are selected from acrylate, methacrylates and norbornene.

The self-surface-activating amphiphilic protein or self-surface-activating amphiphilic peptide may be crosslinked after assembly at the oil/lipid-water interface with a crosslinker having two, three, four or more crosslinking moieties, such as an oligofunctional crosslinker or a polyfunctional crosslinker.

The succinimide may be selected from succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester.

The maleimide may be selected from succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), PEG-bis maleimide, PEG-tetra arm maleimide or other maleimide-functionalised polymers.

The self-surface-activating amphiphilic protein or self-surface-activating amphiphilic peptide may be crosslinked with crosslinks provided by covalent or supramolecular bonds (hydrogen bonding, host-guest interactions, electrostatic interactions or pi-pi stacking).

The self-surface-activating amphiphilic protein or self-surface-activating amphiphilic peptide may be crosslinked with crosslinks comprising ester, amide, urethane, carbonate, ether, amine, thioether, and/or thioester moieties.

The conditioning layer may consist essentially of the self-surface-activating amphiphilic protein or self-surface-activating amphiphilic peptide and cross linkers described above. The conditioning layer may consist of the self-surface-activating amphiphilic protein or self-surface-activating amphiphilic peptide and cross linkers described above. For example, the method may further comprise washing to remove other, non-cross-linked, components.

In suitable implementations, the self-surface-activating amphiphilic-protein and/or self-surface-activating amphiphilic-peptide suitable for use in the present disclosure are water soluble, have low tensioactivity and do not display micelle formation (e.g. above a critical micelle concentration).

The self-surface-activating amphiphilic-protein and/or self-surface-activating amphiphilic-peptide may be selected from the group consisting of albumins (e.g. bovine serum albumin, BSA; human serum albumin, HSA; plant based albumin including sunflower albumin and rice albumin; lactalbumin), caseins (e.g. α-casein or β-casein), lactoglobulins (e.g. β-lactoglobulin, βLG), fibrinogen, gelatine (denatured collagen), collagen, fibronectin, laminin, vitronectin, proteins contained in Matrigel, Geltrex or comparable cell adhesive protein formulations, hydrophobins, lysozymes, zein, heliantinin (11S), pea proteins, soy proteins, other plant-based globulins and combinations thereof. These are optionally selected from albumins, lactoglobulins and fibrinogen, and combinations thereof.

The self-surface-activating amphiphilic-protein and/or self-surface-activating amphiphilic-peptide may be selected from the group consisting of albumins (e.g. bovine serum albumin, BSA; human serum albumin, HSA; plant based albumin including sunflower albumin and rice albumin; lactalbumin), caseins (e.g. α-casein or β-casein), lactoglobulins (e.g. β-lactoglobulin, βLG), fibrinogen, gelatine (denatured collagen), hydrophobins, lysozymes, zein, heliantinin (11S), and combinations thereof. These are optionally selected from albumins, lactoglobulins and fibrinogen, and combinations thereof.

The self-surface-activating amphiphilic-protein and/or self-surface-activating amphiphilic-peptide may be sourced from natural origins, or produced recombinantly, with or without modifications. For example, the self-surface-activating amphiphilic-protein may be a natural protein, recombinant protein (e.g. recombinant albumin, casein and β-lactoglobulin), engineered recombinant protein or chemically modified protein. Engineering may allow for crosslinking and/or cell adhesion, or binding of other cell membrane receptors.

The self-surface-activating amphiphilic-protein and/or self-surface-activating amphiphilic-peptide may be sourced from natural origins, or produced recombinantly, with or without modifications. For example, the self-surface-activating amphiphilic-protein may be a natural protein, recombinant protein (e.g. recombinant albumin), engineered recombinant protein or chemically modified protein. Engineering may allow for crosslinking and/or adhesion.

The self-surface-activating amphiphilic-protein and/or self-surface-activating amphiphilic-peptide may display cell-adhesive and crosslinkable moieties (as discussed herein), e.g. they may be recombinantly engineered to display such moieties. The self-surface-activating amphiphilic-protein and/or self-surface-activating amphiphilic-peptide may display RGD, YIGSR, IKVAV, GFOGER and PHSRN or other sequences that can bind integrin receptors.

The self-surface-activating amphiphilic-protein and/or self-surface-activating amphiphilic-peptide may be sourced from plant matter, such as a chickpea, lentil, pea, sunflower, soy, flaxseed and/or wheat protein extract).

The self-surface-activating amphiphilic protein and/or self-surface-activating amphiphilic peptide may be provided at a concentration of:
at most about 500 mg/mL, optionally at most 200 mg/mL, optionally at most about 100 mg/mL; and/or
at least about 10 ng/mL, optionally at least about 0.0001 mg/mL, optionally at least about 0.00075 mg/mL, optionally at least 0.001 mg/mL; and/or
about 10 ng/mL to 500 mg/mL, optionally about 100 ng/mL to 200 mg/mL, optionally about 100 ng/mL to 100 mg/mL, optionally about 1 mg/mL to 100 mg/mL.

Here, the concentration is based on the amount of protein/peptide in the aqueous buffer or cell culture medium when they are deposited. In other words, when preparing a cell culture system by mixing together the various components (aqueous cell culture medium, oil/lipid phase, etc.), the concentration above refers to the amount of protein/peptide mixed/added to the remainder of the aqueous buffer or cell culture medium.

The self-surface-activating amphiphilic protein or self-surface-activating amphiphilic peptide may spontaneously assemble(s) at the liquid-liquid interface.

The cell culture system may be washed after assembly of the interface, e.g. with an aqueous wash such as deionised water, phosphate buffer saline or any other buffer or relevant electrolyte solution. For example, the method may comprise an active step of washing the cell culture system, e.g. with phosphate buffer saline. In some implementations washing is conducted prior to cross-linking. The presence of excess protein/peptide (e.g. non-interfacial protein/peptide, e.g. in a system prior to washing), would otherwise cause deleterious effects during cross-linking (e.g. since excess, non-interfacial protein/peptide would become cross-inked). In some implementations, washing is conducted after cross-linking (e.g. to remove unreacted cross-linkers).

The self-surface-activating amphiphilic protein and/or self-surface-activating amphiphilic peptide may be provided at a concentration of:
at most about 10 mg/cm², optionally at most 5 mg/cm², optionally at most about 1 mg/cm²; and/or
at least about 1 ng/cm², optionally at least about 10 ng/cm², optionally at least about 100 ng/cm², optionally at least 1 μg/cm²; and/or
about 10 ng/cm² to 50 μg/cm², optionally about 20 ng/cm² to 20 μg/cm², optionally about 50 ng/cm² to 10 μg/cm², optionally about 100 ng/cm² to 5 μg/cm².

Here, the concentration is based on the amount of protein/peptide in the aqueous cell culture medium after washing.

The conditioning layer may further comprise a polysaccharide (such as hyaluronic acid or pectin), wherein the polysaccharide is cross-linked with the self-surface-activating amphiphilic protein and/or self-surface-activating amphiphilic peptide.

The polysaccharide may be hyaluronic acid when the self-surface-activating amphiphilic protein and/or self-surface-activating amphiphilic peptide is albumin. The polysaccharide may be pectin when the self-surface-activating amphiphilic protein and/or self-surface-activating amphiphilic peptide is lysozyme.

Crosslinks are formed in a similar manner as discussed above in the context of the self-surface-activating amphiphilic protein and/or self-surface-activating amphiphilic peptide (e.g. by denaturation, and/or after assembly, and/or using the same crosslinker moieties, and/or using the same crosslinkers, and/or using the same crosslinks/interactions, etc., mutatis mutandis).

The conditioning layer may consist essentially of (optionally consists of) the self-surface-activating amphiphilic protein or self-surface-activating amphiphilic peptide, polysaccharides and cross linkers (e.g. wherein the method comprises washing to remove other, non-cross-linked, components).

### Oil or Lipid Phase

In implementations, there is substantially no self-surface-activating amphiphilic protein or self-surface-activating amphiphilic peptide (optionally no protein or peptide) in the oil/lipid phase. In other words, substantially all self-surface-activating amphiphilic protein or self-surface-activating amphiphilic peptide is in the aqueous cell culture medium.

The choice of oil/lipid will depend on a number of factors, such as the type of cells that are to be cultured or the application the cells products are intended for. The oil may be selected from the group consisting of silicone oil, fluorinated oil, hydrocarbons, paraffin oil, mineral oil, fatty acid oils, or a plant-based oil optionally selected from peanut oil, sunflower oil, flax seed oil, soybean oil, castor oil, palm oil, rapeseed oil and olive oil.

The oil may be a silicone oil, such as polydimethylsiloxane (PDMS) or an associated derivative. The successful use of silicone oils, for example when used in the absence of PFBC and other non-fluorinated acyl chlorides, is particularly surprising since cell culture systems of the art where not able to establish proliferation of cells such as stem cells using silicone oils without the use of surfactant small molecules (e.g. Keese & Giaever, Science, 219:1448-1449, 1983 and Keese & Giaever, Proc. Natl. Acad. Sci., 80:5622-5626, 1983).

The oil may be a fluorinated oil. Optionally, the fluorinated oil comprises a perfluorinated aliphatic group (optionally an alkane, such as a branched alkane). The fluorinated oil may (alternatively or additionally) comprise an alkoxy group. In some implementations, the fluorinated oil is an alkoxyperfluoroalkane. The fluorinated oil may be 3-ethoxyperfluoro(2-methylhexane), Novec 7500, hexane, 3-ethoxy-1,1,1,2,3,4,4,5,5,6,6,6-dodecafluoro-2-(trifluoromethyl), 2-(trifluoromethyl)-3-ethoxydodecafluorohexane, FC-40 or perfluorodecalin.

In implementations where a fluorinated oil is used, the oil may be Novec 7500 or FC-40.

### Novec 7500 is hexane, 3-ethoxy-1,1,1,2,3,4,4,5,5,6,6,6-dodecafluoro-2-(trifluoromethyl) (also known as 2-(Trifluoromethyl)-3-ethoxydodecafluorohexane, CAS No. 297730-93-9)

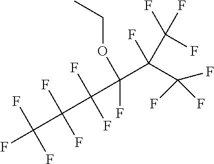 [SMILES: CCOC(F)(C(F)(F)C(F)(F)C(F)(F)F)C(F)(C(F)(F)F)C(F)(F)F]

### FC-40 (also Known as Fluorinert™ FC-40) is a Mixture of 1,1,2,2,3,3,4,4,4-nonafluoro-N,N-bis(1,1,2,2,3,3,4,4,4-nonafluorobutyl)butan-1-amine and 1,1,2,2,3,3,4,4,4-nonafluoro-N-(1,1,2,2,3,3,4,4,4-nonafluorobutyl)-N-(trifluoromethyl)butan-1-amine, CAS No. 51142-49-5)

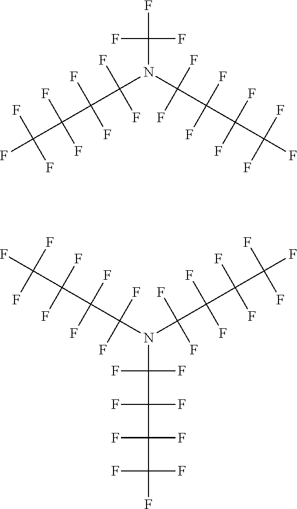 [SMILES: FC(F)(F)N(C(F)(F)C(F)(F)C(F)(F)C(F)(F)F)C(F)(F)C(F)(F)C(F)(F)C(F)(F)F ; FC(F)(F)C(F)(F)C(F)(F)C(F)(F)N(C(F)(F)C(F)(F)C(F)(F)C(F)(F)F)C(F)(F)C(F)(F)C(F)(F)C(F)(F)F]

The oil may be a mineral oil. A mineral oil may be a colourless, odourless, light mixture of higher alkanes from a mineral source, for example a distillate of petroleum. Mineral oils include, but are not limited to, oils known as liquid petroleum, paraffinum liquidum, liquid paraffin, paraffin oil and white oil. The mineral oil may be hexadecane or other aliphatic linear or branched alkanes of comparable molar masses, or with different molar masses.

The oil may be plant oil, optionally rapeseed oil, peanut oil, sunflower oil, flax seed oil, soybean oil or castor oil.

The oil may be plant oil, optionally rapeseed oil, peanut oil, sunflower oil, flax seed oil, soybean oil.

The lipid may be selected from saturated fatty acids, monounsaturated fatty acids, polyunsaturated fatty acids, palmitic acid, lauric acid, myristic acid, stearic acid, oleic acid, eicosenoic acid, docosahexaenoic acid, linoleic acid, or a corresponding ester thereof, or combinations of these lipids.

In some implementations, the cell culture system is an emulsion (in particular, an oil-in-water emulsion). In such implementations, the oil/lipid is present as a plurality of droplets contained within the aqueous cell culture media. The droplets may be microdroplets. Such implementations enable a large number of cells to be cultured by providing a high surface area on which the cells can be cultured. The droplets may be from about 0.1 to about 1000 μm in diameter.

In other implementations, the oil/lipid phase and the aqueous media are a substantially planar sheet at the interface of the oil/lipid and aqueous phases. Such implementations are useful when sheets of cells are desired for a given intended use. The planar sheet may have a surface area of at least 10 cm².

### Other Components

In some implementations, a protein or peptide layer is provided between the conditioning layer and the aqueous cell culture medium. The protein or peptide layer may comprise an extra-cellular matrix (ECM) protein or macromolecule mimicking the cell adhesive properties of ECM proteins. Relevant ECM proteins include: fibronectin, vitronectin, collagen, laminin, agrin, fibroin and elastin. Natural ECM proteins are inherently presenting cell adhesive peptidic domains. Cell adhesive peptide sequences include RGD, YIGSR, IKVAV, GFOGER and PHSRN or other sequences that can bind integrin receptors.

Therefore, in an implementation, the protein or peptide layer comprises a protein selected from the group consisting of extra-cellular matrix (ECM) proteins and macromolecules mimicking the cell adhesive properties of ECM proteins, wherein the macromolecule comprises a cell adhesive peptide sequence, for example a cell adhesive peptide sequence selected from the group consisting of RGD, YIGSR, IKVAV, GFOGER and PHSRN or other sequences that can bind integrin receptors.

In some implementations, the ECM protein is a protein selected from the group consisting of fibronectin, laminin, collagen, vitronectin, agrin, elastin and fibroin and functional fragments thereof. In preferred implementations, the protein comprises collagen or fibronectin. In more preferred implementations, the protein layer consists of collagen and/or fibronectin.

The protein or peptide layer between the conditioning layer and the aqueous cell culture medium may be provided at a concentration of:
at least 0.001 mg/mL, optionally at least about 0.01 mg/mL, optionally at least about 0.1 mg/mL, optionally at least about 1 mg/mL; and/or
at most about 100 mg/mL, optionally at most 10 mg/mL; and/or about 0.001 to 100 mg/mL, optionally about 0.001 to 10 mg/mL, optionally about 0.01 to 1 mg/mL or 0.01 to 1 mg/mL.

Here, the concentration is based on an amount of protein or peptide added to the aqueous cell culture medium to form the layer between the conditioning layer and the aqueous cell culture medium.

The method may further comprise incubation in a crosslinking protease solution, such as thrombin, after assembly of the self-surface-activating amphiphilic protein or self-surface-activating amphiphilic peptide at the liquid-liquid interface. Proteases such as thrombin behave as a catalyst. For example, thrombin may cleave part of fibrinogen, exposing a fragment that was previously shielded and that can now interact with other domains within fibrinogen, resulting in crosslinking.

The protease may be provided at a concentration of at least about 0.1 units/mL, optionally at least about 0.25 units/mL, optionally at least about 0.5 units/mL.

### Types of Cells to be Cultured

The cell culture systems and methods of the disclosure are suitable for the culture of adherent cells. The systems and methods of the disclosure are particularly suited for the culture of stem cells, more particularly adherent stem cells.

In some implementations the cells will be human cells, although other cell types can also be used with the present disclosure (in particular mammalian cells, such as equine, canine, porcine, bovine, ovine, or rodent (e.g., mouse or rat) cells).

The cells may be engineered cells, e.g. to express protein, ECM proteins, antibodies, enzymes, hormones, growth factors, exosomes, viral particles, etc. The cells may be selected from a group consisting of human primary keratinocytes (HPKs), mesenchymal stem cells (MSCs), induced pluripotent stem cells (iPSCs), Chinese hamster ovary (CHO) cells, human umbilical vein endothelial cells (HUVECs), adipose derived stem cells, amniotic fluid derived stem cells, hepatocytes, lung epithelial cells, cord blood stem cells, fibroblasts, HEK293 cells, cardiomyocytes and skeletal muscle cells.

The cells may be:
human primary keratinocytes; or
mesenchymal stem cells; or
induced pluripotent stem cells.

In an implementation of the disclosure, the cells are induced pluripotent stem cells (iPSCs). Induced pluripotent stem cells can be directly generated from adult cells, can propagate indefinitely, and are capable of differentiating into any cell type in the body.

### Interfacial Properties

An additional and important consideration of the selection of the components on the cell culture system are the mechanical properties of the resulting cell culture support at the liquid-liquid interface. The present inventors have surprisingly found that the cell culture systems of the disclosure provide a surface having a suitable elasticity that enables the long-term culture of adherent cells, including stem cells. The investigations by the inventors surprisingly found that cell proliferation is better correlated with the level of elasticity (stress retention) than with maximum stress (stiffness) of the conditioning layer. The combinations used in the present disclosure to form the conditioning layer provide a rigid nanoscale quasi 2D-material that can support the culture of adherent cells.

An important mechanical property of the cell culture systems is the elasticity of the liquid-liquid interface. The elasticity of this interface is controlled by the conditioning layer. The elasticity of the interface is sufficient to enable adherent cells (such as adherent stem cells) to proliferate to at least about 50% confluency. The measure of elasticity is a measure of the degree of elasticity of the interface between the aqueous and oil/lipid phases of the cell culture system.

The elasticity of the interface (% of stress retained at the interface in stress relaxation experiments) may be between about 30% and about 100%, or about 40% and about 100%, or about 50% and about 100%, or about 60% and about 100%, or about 65% and about 90%, or about 65% and about 80%. The elasticity of the interface may be at least about 60%.

The elasticity of the interface (% of stress retained at the interface in stress relaxation experiments) may be between about 60% and about 100%, or about 65% and about 90%, or about 65% and about 80%. The elasticity of the interface may be at least about 60%.

When elasticity is at least about 30% (e.g. at least about 60%), the culture of dense adherent cell colonies is possible.

Elasticity can be measured according to any suitable method known to the skilled person. One such method is the use of a rheometer. For example, one method may use a rheometer fitted with an interfacial rheology system to allow stress-relaxation experiments to be carried out. In such set up, a Du Noüy ring is fitted to the shaft of the rheometer and oscillates. The percentage elasticity can be defined as the level of stress retained at infinite time (extracted from curve fitting), compared to the stress exerted on the sample just before relaxation is allowed to start. Similar experiments can be carried out with an oscillating magnetic bar positioned at the interface between the two liquids, using a magnetic rig to monitor deformations.

Accordingly, in an implementation, the elasticity of the liquid-liquid interface is at least about 30% (e.g. at least about 60%) as measured in a stress-relaxation experiment using a rheometer. In another implementation of the disclosure, the elasticity of the liquid-liquid interface is at least about 60% as measured using a Du Noüy ring tensiometer.

One detailed method to quantify interfacial stress relaxation is as follows. Rheological measurements were carried out on a hybrid rheometer (DHR-3) from TA Instruments fitted with a double wall ring (DWR) geometry and a Delrin trough with a circular channel. The double wall ring used for this geometry has a radius of 34.5 mm and the thickness of the Platinum-Iridium wire is 1 mm. The diamond-shaped cross-section of the geometry's ring provides the capability to pin directly onto the interface between two liquids and measure the interface properties without complicated sub-phase correction. 19 mL of the fluorinated oil were placed in the Delrin trough and the ring was lowered, ensuring contact with the surface, via an axial force procedure. The measured position was set 500 μm lower than the contact point of the ring with the oil-phase surface. Thereafter, 15 ml of the PBS buffer were carefully syringed on top of the oil phase. Stress relaxation tests were performed during 120 s, with a strain rate of 0.5%/s and 1%/s, for maximum strains of 0.5% and 1%, respectively.

After locating the maximum stress, the stress relaxation data was fit to a double exponential decay function:

$$y = A 1 \times exp ( - \frac{x}{t 1} ) + A 2 \times exp ( - \frac{x}{t 2} ) + y 0 .$$

Stress retention (in % of the maximum stress) was calculated by equation:

$$Stress retention \% = \frac{y 0}{y 0 + A 1 + A 2} \times 100 \%$$

The thickness of the conditioning layer can also influence its suitability for culturing adherent cells. The dry thickness of the conditioning layer may be from about 1 nm to about 10 μm, from about 1 nm to about 100 nm, from about 1 nm to about 50 nm and from about 1 nm to about 20 nm. The thickness is the dry thickness of the layer and can be measured by atomic force microscopy, for example. Swollen thicknesses can be measured using interometry, 3D optical profiling, neutron reflectivity or ellipsometry.

The cell culture system may be in the form of an emulsion or in the form of a planar sheet, optionally an emulsion.

The shear interfacial modulus of the interface between the aqueous medium and the functionalised oil/lipid phase may be at least about 0.001 N/m, optionally at least about 0.01 N/m.

The pH of the aqueous medium during formation of the conditioning layer may be from about 3 to about 13, or from about 4 to about 12, or from about 5 to about 12, or from about 6 to about 11.

The elasticity of the cell conditioning layer may be at least about 60%, the shear interfacial modulus of the interface between the aqueous medium and the oil/lipid phase may be at least about 0.001 N/m, optionally at least about 0.01 N/m, and the pH of the cell conditioning layer may be from about 6 to about 11.

### Aqueous Cell Culture Medium

The aqueous cell culture medium can be any suitable cell culture medium in the art useful in the culture of adherent cells, and the skilled person will be aware of appropriate cell culture media and will be able to choose an appropriate cell culture media for the culture of a given population of cells. The choice of cell culture medium will be familiar to the person of skill in the art and will depend on the type of cells being cultured. Advantageously, the cell culture systems of the disclosure can use the same cell culture medium as would be used for a given cell type if it was being grown on a solid substrate.

For example, the aqueous cell culture media may comprise a carbon source, various salts and optionally a source of amino acids and/or nitrogen. The media may be a chemically defined media (in which all of the components or known), or an undefined culture media may be used, which comprise yeast, animal or plant extracts (such as BSA).

Depending on the context, the cell culture system may comprise selective media, differential (i.e. differentiation-inducing) media, transport media, cell-sorting media, or enriched media. Again, the appropriate choice of media would be apparent to the skilled person for a given context (cell type, assay, length of culture etc. Examples of suitable cell culture media for the culture of adherent cells, including stem cells, include DMEM (many different cell types), FAD (for example for keratinocytes), KSFM (for example for keratinocytes) and EBM-2 (for example for HUVECs), and there are many more available that could be used in the cell culture systems of the disclosure.

The aqueous cell culture medium may be sterile. When culturing cells, the cell culture medium may be replaced or replenished to allow the longer-term culture of cells.

In one aspect of the disclosure, the cell culture system is suitable for use in a bioreactor, in particular a 3D bioreactor. The bioreactor contains the cell culture system of the disclosure and a culture of adherent cells, such as adherent stem cells. The cells adhere to the liquid-liquid interface. The bioreactor can take any suitable form, for example the bioreactor may be a cell culture flask or bag.

### Methods of Culturing Cells

The present disclosure provides a method of culturing adherent cells comprising culturing the cells in a cell culture system of the disclosure. Methods of culturing cells are also referred to herein as methods of expanding a cell population, since a cultured cell population will be expanded by the method. The cell cultures are not suspension cultures as the cells are adhered to the conditioning layer of the cell culture system.

Such methods of the disclosure may comprise seeding the cells at the liquid-liquid interface of the cell culture system (in particular, at the protein/peptide layer of the conditioning layer of the system) and contacting the seeded cells with a cell culture medium. The choice of cell culture medium will be particular to the cells being cultured and the skilled person is familiar with which cell media are suitable for which cell types.

The method may be for the long-term culture of cells, such as at least about 1 day, or at least about 5 days, or at least about 7 days, or at least about 14 days, or at least about 25 days. Optionally, the cells are cultured for at least about 7 days. At least 80% of the cells may be alive at the end of the culture period.

The method may further comprise seeding the cells at the liquid-liquid interface of the cell culture system.

The method may further comprise harvesting the cultured cells from the cell culture system. This can be achieved by any suitable method, for example centrifugation (for example at 1200 rpm for 5 min, but not exclusively), by transferring cells to another substrate, by allowing the oil to evaporate, or by using a chemical or enzyme that allows to partially degrade the protein and/or peptide that are present at the interface.

One of the advantages of the present disclosure is that the methods used to harvest the cells do not require enzymatic digestion or treatment at low temperatures, which are significant drawbacks of current culture methods that grow cells on solid substrates.

Alternatively, the cultured cells can be used without being harvested from the cell culture system. For example, when the cell culture system is in the form of an emulsion, the method may comprise administering the emulsion containing the cultured cells directly to a patient or to a tissue engineering platform. In an implementation of the disclosure, the method comprises administering a culture of cells to a hydrogel. Such implementations are useful for tissue engineering such as 3D tissue engineering.

### Methods of Preparation of the Cell Culture Systems

The present disclosure also provides a method of production of the cell culture systems of the disclosure. These can be assembled by simply placing in contact the oil/lipid phase and the aqueous phase (including suitable self-surface-activating amphiphilic protein or self-surface-activating amphiphilic peptide) and agitating vigorously to create an emulsion. Alternatively, this can be done in a more controlled way using a microdroplet or picodroplet fabrication method or other similar systems allowing the formation of emulsions, including using microdroplet microfluidic systems, Couette cells or membrane emulsification systems, for example.

In one implementation of the disclosure, the method of production of the cell culture system comprises contacting the chosen oil/lipid and self-surface-activating amphiphilic protein/peptide with the aqueous medium and forming an oil/lipid-in-water emulsion. The step of forming an oil/lipid-in-water emulsion may comprise, for example, shaking the mixture containing the components of the cell culture system. Alternatively, the emulsion may be formed using microdroplet or picodroplet fabrication platforms, including using microdroplet microfluidic systems, Couette cells or membrane emulsification systems, for example.

2D interfaces can be generated by sequential incubation and washing steps (no step of forming an emulsion is required when the cell culture system is a sheet). Accordingly, in one implementation of the disclosure, the method comprises contacting the chosen oil/lipid and components of the conditioning layer with a first aqueous medium and incubating the components to allow a planar interface to be formed between the two components. The cell culture system is then washed.

According to a second aspect, therefore, provided herein is a culture system for the culture of adherent cells at a liquid-liquid interface, the culture system comprising:
a. an aqueous cell culture medium; and
b. an oil or lipid phase, wherein the oil/lipid phase is functionalised with a conditioning layer that is disposed between the aqueous cell culture medium and the oil/lipid phase and the conditioning layer comprises a self-surface-activating amphiphilic protein or self-surface-activating amphiphilic peptide layer;
the system comprising culturing the adherent cells at the interface between the oil/lipid phase and the aqueous cell culture medium.

### Additional Implementations

According to a third aspect, provided herein is a use of the culture system defined in the second aspect for the culture of adherent cells at a liquid-liquid interface.

For examples, cells grown according to the methods of the disclosure may be useful in tissue engineering, such as bone regeneration, wound healing, cartilage regeneration, tendon regeneration and cardiac repair (for example, in the treatment of myocardial infarction). The desired cells, e.g. stem cells, can be cultured according to the method of the disclosure, including possible differentiation of the cells, harvested, and then applied to a patient in a suitable manner, such as in the form of a bioengineered tissue construct. The tissue construct can be formed into an appropriate shape or arrangement according to its purpose. For example, in the case of cells grown in sheets according to a method of the disclosure, said cell sheets may be applied directly. Alternatively, cells cultured according to methods of the present disclosure may be applied to a 3D scaffold. Suitable scaffolds are biocompatible and may be biodegradable such that the scaffold is slowly degraded after implantation into a patient. The scaffolds will have mechanical properties that are suitable for the intended purpose. Suitable scaffolds include hydrogel or collagen scaffolds. Suitable tissue engineering techniques that employ scaffolds are discussed in, for example, O'Brien, Materials Today, 14(3):88-95, 2011.

Cells cultured according to methods of the disclosure may be used for the generation of biotherapeutics such as extra-cellular vesicles and exosomes, or the generation of proteins, such as extra-cellular matrix proteins, growth factors, cytokines, therapeutic peptides, microRNA or antibodies.

The present disclosure therefore also provides a method of producing a protein or other molecule of interest, comprising culturing a cell according to a cell culture method of the disclosure, wherein the cell expresses or produces the protein or molecule of interest, and collecting the protein or molecule of interest from the cell culture medium. The cells may have been transfected or otherwise engineered to product the protein or molecule of interest. For example, the cells may have been transfected with vectors encoding for a protein of interest. The sequence encoding the protein is operable linked to a promoter that is compatible with the cell being transfected. In some implementations of the disclosure, the method includes the step of transfecting the cell with the plasmid encoding the protein of interest.

For example, in the case of antibody production, a cell may be transfected with two plasmids, one plasmid encoding a heavy chain of an antibody and the other plasmid encoding a corresponding light chain of an antibody, wherein the sequences encoding the heavy and light chains are operably linked to promoters that compatible with the cell being transfected (such as a CMV promoter). The cells express the sequences encoding the antibodies and via post-translational modification secrete the assembled antibody into the cell culture medium. The antibody can then be extracted from the cell culture medium in the usual way.

An appropriate cell type can be used to generate the protein or other molecule of interest. For example, although the methods of the disclosure are particularly suited for the culture of stem cells, other adherent cells may be useful in the contest of the generation of proteins (such as antibodies). CHO, HEK293 and Cos7 cells may be of particular use for this purpose.

The disclosure also provides various methods of treatment using cell populations cultured or expanded according to a method of the disclosure. For example, the disclosure is useful in the expansion of cell populations in cell therapy (e.g. stem cell therapy, including allogenic and autologous stem cell therapy).

In one implementation of the disclosure, there is provided a method of culturing a population of adherent cells according to a method described herein, obtaining an expanded population of cells, and administering the expanded population of cells to a patient. In autologous cell therapy (e.g. stem cell therapy), the donor and recipients of the cells are the same patient. The cells may be obtained by any suitable means known to the skilled person, for example the isolation of cells (e.g. stem cells) from a patient sample such as a patient's bone marrow. Once isolated, the cells can then be washed and seeded onto a cell culture system of the disclosure and expanded until a suitable confluency is reached.

The present disclosure also provides methods for the purification of adherent cells, including adherent stem cells. In such cases, cell mixtures (e.g. obtained from bone marrow or adipose tissue aspirates) are directly placed in contact with oil/lipid emulsions functionalised with interfaces and briefly incubated (for example 20 min to 1 h) before separating the remaining cells from the emulsions (with bounded purified cells). This separation can be carried out via sedimentation or simple centrifugation (1200 rpm for 3-5 min).

Accordingly, in one implementation of the disclosure there is provided a method of purifying adherent cells, comprising:
a) providing a mixture of cells obtained from a patient, wherein the mixture of cells comprises a cell population of interest;
b) contacting the cells with a cell culture system of the disclosure;
c) incubating the cells in the cell culture system;
d) separating cells of the cell population of interest from the remaining cells in the mixture.

The mixture of cells maybe a cell sample from a patient that is obtained and requires purification to obtain a purified cell population of interest. The sample may be a sample that comprises stem cells. In one implementation, the sample may be a bone marrow sample or an adipose tissue sample (for example a bone marrow or adipose tissue aspirate).

The step of contacting the cells with a cell culture system of the disclosure may comprise seeding the mixture of cells onto the cell culture substrate (i.e. the conditioning layer) of the cell culture system (i.e. the surface of the oil/lipid). Cell culture systems in the form of emulsions are of particular use in the methods of purification as they provide a larger surface area for capturing the cells of the cell population of interest and it is easier to mix the contents of the patient sample with emulsions (for example, they can be mixed by simple shaking).

The step of incubation can be carried out for a sufficient time to allow the cells to adhere to the conditioning layer of the cell culture system. For example, the step of incubation may comprise incubation of the cells in the cell culture medium for at least 10 minutes. This allows cells of the cell population of interest to adhere to the conditioning layer.

The step of separating the cells of the cell population of interest from the remaining cells in the mixture may comprise removing the oil/lipid with conditioning layer and adhered cells from the cell culture system. This can be achieved by, for example, centrifugation or sedimentation (or other suitable methods known to the skilled person). In this way, a purified population of cells can be provided.

The method may further comprise a step of culturing the captured cells in the cell culture system. This enables the population of adhered cells to expand. Of course, the cells can be later harvested from the cell culture system as well, as described elsewhere.

The cell population of interest is an adherent cell population. The cell population of interest may be a population of stem cells. For example, if the sample was an adipose tissue sample or a bone marrow sample, the cell population of interest may be a population of mesenchymal stem cells.

Purification of cells may be further improved by including a ligand or antibody that promotes adhesion of cells belonging to the cell population of interest. Example such ligands include protein A or protein G, or a combination thereof, optionally with albumin. The ligand or antibody may be incorporated into the cell culture system when the cell culture system is manufactured, to provide cell culture system that comprise the ligand for the cell population of interest. The ligand or antibody can be incorporated into the cell culture system by any suitable means, for example by directly adsorbing the ligand, adsorbing a first polymer layer (PLL or other cationic polymer, for example), followed by adsorption of the ligand, or by forming a biotinylated polymer layer (for example based on PLL-PEG-biotin), followed by streptavidin binding and capture of a biotinylated ligand.

In a more specific implementation, the method of purification may comprise mixing the patient sample with a cell culture system of the disclosure that is an emulsion, for example by shaking. To promote adherence of the cell population of interest to the cell culture system, the emulsion may include a ligand that is specific for the desired cell type (for example an antibody that is specific to a cell surface marker present in the desired cell type). The emulsion comprising the adhered cells can then be separated from the remaining contents of the cell sample, for example by centrifugation, to allow the cell population of interest to be cultured using the cell culture system of the disclosure.

Methods of purification and culture of cells according to a method of the disclosure may promote the expression of stem cell markers in cultured stem cells or purified stem cell populations. The stem cells may also exhibit low levels of expression of differentiation markers (such as OCN and ALP). Without wishing to be bound by theory, the inventors hypothesise this is due to a selected effect of the culture of cells on non-flat liquid-liquid interfaces as stems cells with low stem cell surface markers (lower stem cell potential) are not able to adhere as efficiently to the liquid-liquid interface and therefore are selected out. Therefore, the methods of the disclosure provide a general and simple mechanism to sort cells, replacing other sorting technologies such as fluorescence activated cell sorting (FACS) and magnetic-activated cell sorting (MACS). In addition, it is hypothesised that selection of “better” stem cells can be achieved via initial adhesion of cells expression the highest levels of integrins and stem cell markers.

The present disclosure also provides the use of the cell culture systems of the disclosure in the purification of an adherent cell population.

According to a fourth aspect, provided herein is a method of expanding a population of adherent cells comprising culturing the cells at a liquid-liquid interface according to the first aspect and harvesting the cells from the culture medium.

The adherent cells may be cultured to at least 30% confluence prior to harvesting.

According to a fifth aspect, provided herein is a bioreactor comprising a culture of adherent cells (optionally stem cells), wherein the stem cells adhere to a liquid-liquid interface in a cell culture medium defined in the second aspect.

The cells (e.g. stem cells) may be at 30% confluence or above.

The bioreactor may be a cell culture flask or bag.

According to a sixth aspect, provided herein is a kit of parts comprising combinations of one or more oils or lipids, and one or more self-surface-activating amphiphilic proteins or self-surface-activating peptides for the culture of adherent cell and optionally stem cells, optionally wherein the oil/lipid(s) and self-surface-activating amphiphilic protein(s)/self-surface-activating amphiphilic peptide(s) are as defined in the first aspect.

The present disclosure also provides populations of cells that have been cultured or expanded according to a methods described herein.

All features discussed herein in respect of any of the methods, uses, systems, apparatuses, kits or products (first to sixth aspects) relate to all other methods, uses, systems, apparatuses, kits or products mutatis mutandis.

The present disclosure will now be further exemplified by reference to a number of specific examples, which are not intended to be limited on the scope of the disclosure.

### EXAMPLES

### Materials and Methods

Materials and Chemicals. Native (Sigma), anionic and cationic BSA were used as received or prepared as described in (Zhong et al., 2020). The fluorinated surfactant 2,3,4,5,6,-Perfluorobenzoyl chloride, PBS, Trichloro (1H,1H,2H,2H-perfluorooctyl) silane (97%) and the 1H,1H,2H,2H-Perfluorodecanethiol (97%) were purchased from Sigma Aldrich Co. The fluorinated oil (Novec 7500; 3M) is from ACOTA. The SPR-Au chips were obtained from Ssens. Propylene glycol monomethyl ether acetate (PGMEA) is from Sigma-Aldrich Co; 484431. Fibrinogen from bovine plasma is from Sigma-Aldrich; 341573.

Human Primary Keratinocyte cell line culture and seeding. Human primary keratinocytes were cultured in Keratinocytes Basal Medium 2 (KBM2, PromoCell). For proliferation assays, HPK cells were harvested with trypsin (0.25%) and versene solutions (ThermoFisher Scientific, 0.2 g/L EDTA Na₄ in Phosphate Buffered Saline) at a ratio of 1/9. Cells were then resuspended with differentiation medium (FAD) prepared with DMEM/F12 (1:1) (1×) and DMEM (ThermoFisher Scientific) at a ratio of 1:1, containing 1% L-Glutamine (200 mM), 1% Penicillin-Streptomycin (5,000 U/mL), 0.1% insulin, 0.1% Hydrocortisone equivalent (HCE) and 10% of Fetal Bovine Serum (FBS, Labtech). HPK cells were centrifuged for 5 min at 1200 rpm, counted and resuspended in KBM2, at the desired density before seeding onto substrates. Cells were left to adhere and proliferate in an incubator (37° C. and 5% CO2) for different time points (at day three and day seven of culture), prior to staining and imaging. For cell spreading assays, HPK cells were harvested and seeded onto fluorinated droplets at a density of 25,000 cells per well (13,000 cell/cm²). For passaging, cells were reseeded in a preconditioned T75 flask, with collagen type I (20 μL of collagen into 10 mL of PBS for 20 min), at a density of 250,000 cells per flask.

Human Bone Marrow Derived Mesenchymal Stem Cell culture and seeding. hBM-MSCs were purchased from PromoCell and cultured according to the supplier's recommendations.

Human Induced Pluripotent Stem Cell culture and seeding. HPS10114i-vabj3 (HlipSci) was purchased from UK Health Security Agency. The iPSCs are routinely cultured in Essential 8 (E8) Flex Medium (ThermoFisher Scientific; A2858501) at 37° C. and 5% CO₂. Flasks and well plates are first incubated in vitronectin (VN) solution (10 μg/mL) for 1 hour. For routine passaging, cells were washed with PBS once and incubated in EZ-Lift Stem Cell Passaging Reagent (Sigma-Aldrich; SCM139) for 4 minutes on a horizontal orbital shaker at 37° C. for 4 minutes. Detached cells were than transferred into a tube containing equal amount of E8 Flex medium. The flask was washed with E8 flex medium and transferred to the same tube. The cells were centrifuged at 120×g for 3 minutes. The supernatant was aspirated and resuspended in E8 Flex medium and passaged into flasks (1:4 passaging ratio). For seeding and dissociation into single cells, cells were primed with E8 flex medium containing 10 μM Y27632 (E8-ROCKi) for 1 hour. After 1 hour, cells were washed once with PBS and incubated in Gentle Cell Dissociation Reagent (Stemcell Technologies; 100-0485) for 9 minutes at 37° C. Cell suspension was then pipetted up and down 3 times to dissociate cell clusters into single cells. The cell suspension was transferred into a tube containing equal volume of E8-ROCKi. The flask was washed once with E8-ROCKi and transferred into the same tube. The cell suspension was centrifuged at 120×g for 3 minutes. Cells were then resuspended and counted. The cells were then seeded at the desired seeding density.

Interfacial shear rheology measurements. Interfacial rheological measurements were carried out on a Discovery Hydrid-Rheometer (DHR-3) from TA Instruments, using a Du Nouy Ring geometry and a Delrin trough with a circular channel. The Du Nouy ring has a diamond-shaped cross section that improves positioning at the interface between two liquids to measure interfacial rheological properties whilst minimizing viscous drag from upper and sub-phases. The ring has a radius of 10 mm and is made of a platinum-iridium wire of 0.36 mm thickness. The Derlin trough was filled with 4 mL of fluorinated oil (with or without surfactant). Using axial force monitoring, the ring was positioned at the surface of the fluorinated oil, and was then lowered by a further 200 μm to position the medial plane of the ring at the fluorinated phase interface. 4 mL of the PBS solution were then gently introduced to fully cover the fluorinated sub-phase. Time sweeps were performed at a frequency of 0.1 Hz and temperature of 25° C., with a displacement of 1.0 10⁻³ rad (strain of 0.1%) to follow the self-assembly of the protein nanosheets at corresponding interfaces. In each case, the protein solution (1 mg/mL) was added after 15 min of incubation and continuous acquisition of interfacial rheology data for the naked liquid-liquid interface. Before and after each time sweep, a frequency sweep (with displacements of 1.0 10⁻³ rad) and amplitude sweeps (at a frequency of 0.1 Hz) were carried out to examine the frequency-dependent behavior of corresponding interfaces and to ensure that the selected displacement and frequency selected were within the linear viscoelastic region.

Surface plasmon resonance (SPR). SPR measurements were carried out on a BIACORE X from Biacore AB. SPR chips (SPR-Au 10×12 mm, Ssens) were plasma oxidized for five minutes and then incubated in a 5 mM ethanolic solution of 1H,1H,2H,2H-Perfluorodecanethiol, overnight at room temperature. This created a model fluorinated monolayer mimicking the fluoriphilic properties of Novec 7500. The chips were washed once with water, dried in an air stream and kept dry at room temperature prior to mounting (within a few minutes). Thereafter, the sensor chip was mounted on a plastic support frame and placed in a Biacore protective cassette. The maintenance sensor chip cassette was first placed into the sensor chip port and docked onto the Integrated μ-Fluidic Cartridge (IFC) flow block, prior to priming the system with ethanol. The sample sensor chip cassette was then docked and primed once with PBS. Once the sensor chip primed, the signal was allowed to stabilize to a stable baseline, and the protein solution (1 mg/mL in PBS) was loaded into the IFC sample loop with a micropipette (volume of 50 μL). The sample and buffer flow rates were kept at 10 μL/min throughout. After the injection finished, washing of the surface was carried out in running buffer (PBS) for 10 min. Washing of the surface was allowed to continue for 10 min prior to injection of the second protein (collagen or fibronectin at 10 μg/mL and 100 μg/mL in PBS, respectively; volume of 50 μL), at a flow rate of 10 μL/min. Buffer (PBS) was flown on the sensor chip for 10 min to wash off excess protein solution and data was allowed to continue for a further 10 min.

Generation of fluorinated pinned droplets for cell culture. Thin glass slides (25×60 mm, VWR) were washed with isopropanol and dried under nitrogen, prior to plasma oxidation for 10 min (Henniker Plasma HPT-200). Slides were then placed into an anhydrous ethanol solution (9.5 mL) containing trichloro-1H,1H,2H,2H-perfluorooctyl silane (97%, Sigma-Aldrich) (500 μL) for 1 h, at room temperature. The fluorinated glass slides were cut into chips (1×1 cm) and placed into a 24 well plate (for Hoechst staining), or the glass slides were kept at their original dimensions and embedded on sticky-slide 8 wells plates (Ibidi), for imaging on a confocal microscope. After sterilization with 70% ethanol, the wells were washed (once) and then filled with 2 mL (or 600 μL for the sticky wells) of PBS (pH 7.4 for βLG, Fibrinogen and different BSA types, and pH 10.5 for poly(L-lysine), PLL). 100 μL of fluorinated oil (or 10 μL for the sticky wells), with or without fluorinated surfactant (10 μg/mL) were added to the surface of the glass slide, forming a fluorinated pinned droplet. For samples prepared in 24 well plates, 30 μL of the oil phase was removed using a micropipette, to form a flatter oil droplet. For protein deposition, 10 μL of βLG, BSA solution or Fibrinogen (100 mg/mL) were added into PBS phase contained in the well (final concentration of 1 mg/mL; volume used for Ibidi well was only 8 μL) and incubated for 1 h. After the incubation time, wells were washed six times with PBS (by dilution/aspiration, ensuring the oil surface did not become exposed to air). Fibronectin (final concentration of 10 μg/mL or 25 μg/mL for Fibrinogen samples) or collagen type I (100 μg/mL, final concentration) were added into the PBS solution and incubated for 1 h, followed by washing with PBS (four times) and with medium depending on the cell type (twice). For iPSC seeding, E8-ROCKi was used instead. Medium was changed the next day with E8 flex medium.

Preparation of Flat Interfaces. To prepare flat interfaces, a 24-well plate was plasma treated for 10 minutes (Henniker Plasma HPT-200). Two solutions were prepared. Per well to be treated, 300 μL of ethanol or methanol is mixed with 12 μL of triethylamine. For example, for 12 wells, solution A is prepared by mixing 3.6 mL of anhydrous ethanol or methanol with 144 μL of triethylamine. For solution B, 3.6 mL of ethanol or methanol is mixed with 144 μL of Trichloro(1H,1H,2H,2H-perfluorooctyl) silane instead. After plasma treatment, Solution A was added and shaken lightly to evenly coat the wells. Solution B was then added and immediately sealed with parafilm. The reaction was incubated overnight at room temperature.

To prepare the flat interfaces, the reaction mixture was aspirated, and wells were briefly washed with 70% ethanol. The wells were then washed with PBS three times and left to dry briefly. Novec oil was added at 500 μL per well and 2 mL of PBS was added to each well. For PLL interfaces, PBS pH 10.5 was used instead of pH 7.4. This was then incubated at the CO2 incubator for 20 minutes. The resulting air bubbles were removed, and concentrated protein solutions were added. The stock solution for βLG/Fibrinogen and Poly-L-Lysine is 100 mg/mL and 10 mg/mL respectively. The working concentrations for these two proteins are 1 mg/mL and 200 μg/mL respectively. After 1 hour, the excess protein was washed off with DPBS 6×, leaving behind an estimated 500 μL of aqueous solution. If Sulfo-SMCC is to be added, 500 μL of 2 mg/mL Sulfo-SMCC is added to the resulting interface. Sulfo-SMCC was then washed off 6× with DPBS after 1 hour. If Fibronectin is to be added, a final concentration of 25 μg/mL is added to the resulting interface. The interfaces other than Fibrinogen were then incubated with VN for 1 hour and washed off 3× with E8 flex medium or MSC media. For Fibrinogen interfaces, MSCs were seeded at a final density of 5K per well. Per well, 500 μL of E8 flex medium supplemented with 20 μM Y-27632 was added—resulting in an aqueous solution with E8 Flex medium supplemented with 10 μM Y-27632. Cells were dislodged from their plates, resuspended in E8 flex medium with Y-27632, counted with a haemocytometer, and plated at 40,000 cells per well.

Preparation of fluorinated oil emulsions. Emulsions were generated using 1 mL of fluorinated oil (Novec 7500, 3M) and 2 mL of protein aqueous solution (1 mg/mL of BSA, Fibrinogen, AcBSA, MA-BSA, NB-BSA or βLG in PBS pH7.4), added to a glass vial. The vial was shaken for 15 s and incubated for 1 h at room temperature. The aqueous phase was aspirated and replaced with PBS 6 times. The BSA and βLG nanosheets were crosslinked by incubating the emulsions in 1 mg/mL Sulfosuccinimidyl 4-(N-maleimidomethyl) cyclohexane-1-carboxylate (Sulfo-SMCC) in water for 1 h at room temperature. The AcBSA, MA-BSA and NB-BSA nanosheets were crosslinked at pH10.5 for 3 h at room temperature. The aqueous phase was aspirated and replaced with PBS 6 times. Emulsions were incubated for 1 h at room temperature in Fibronectin (10 μg/mL, or 50 μg/mL for Fibrinogen at pH 7.4) or VN (20 μg/mL, pH 7.4) or RGD peptide (1.6 mg/ml, CGGRGDSPG, pH 8 for NB-BSA, pH 9 for Ac-BSA and MA-BSA,) solutions to functionalise the interface.

Generation of fluorinated oil emulsions with controlled sizes. A microfluidic device was fabricated with a flow focusing channel. The device was generated using standard soft photolithography using SU-8 2050 (Microchem). Briefly, a silicon wafer is cleaned with acetone and isopropanol. The silicon wafer was then dried with nitrogen gas. SU-8 2050 is then spin coated onto the silicon wafer. It was then softbaked on a levelled hotplate. After cooling down, a glass plate with the photomask was placed on top of the spin coated SU-8 and exposed to UV light. The wafer was then baked on a level hotplate. The pattern was then developed in PGMEA with shaking. Once the pattern was clear and white residues are no longer visible, the wafer was cleaned with isopropanol and dried with nitrogen gas. The pattern was further baked at 150° C. for 3 minutes.

PDMS monomer and curing agent were mixed together at a 10:1 ratio. This was then mixed together until opaque. This was then degassed several times in a desiccator and vacuum until the mixture is clear and transparent. The mixture was then poured onto the petri dish containing the wafer. This was then baked at 80° C. for 2 hours. The hardened PDMS was then cut with a scalpel. A biopsy punch (1.5 mm) was used to bore the inlets and outlets on the device. Dust was removed from the device with tape. The device and glass slides were further cleaned with water and isopropanol and dried with nitrogen gas. The device and glass slide were then treated with plasma for 2 minutes at 100% power. The device and glass slide were then pressed onto each other to attach them together. Tygon microbore tubing (Cole-Parmer; WZ-06419-01) was then attached onto the inlets and outlets of the device.

The device was then sterilized with 70% ethanol. To generate the emulsions, βLG solution (1 mg/mL) and fluorinated oil were flowed into the inlets using a pressure controller (Elveflow; OB1 MK3+). The pressure for the aqueous solution was adjusted to control the size of the microdroplets.

Preparation of rapeseed oil emulsions. Emulsions were generated using 1 mL of filtered rapeseed oil (Sainsbury's) and 2 mL of protein aqueous solution (1 mg/mL of AcBSA, MA-BSA or NB-BSA in PBS). The vial was shaken for 15 s and incubate for 1 h at room temperature. The liquid aqueous phase was aspirated and replaced with PBS 6 times. nanosheets were crosslinked at pH 10.5 for 3 h at room temperature. The liquid phase (aqueous) was aspirated and replaced with PBS 6 times. Emulsions were incubated for 1 h at room temperature either in Fibronectin (10 μg/mL, pH 7.4) or RGD peptide (1.6 mg/ml, CGGRGDSPG, pH 8 for NB-BSA, pH 9 for Ac-BSA and MA-BSA,) solutions to functionalise the interface.

Cell seeding on emulsions. HEK293T cells, human iPSCs, Human primary keratinocytes and human MSCs were cultured according to manufacturer's instructions. For cell culture, 500 μL of corresponding medium was transferred per well in a 24 well plate treated with 25 μg/mL poly(L-lysine)-graft-polyethylene glycol (PLL-g-PEG, SuSoS). Then, 100 μL of emulsion were transferred per well. Different cell densities were used depending on the cell type. Being 10⁴ cells/well for HEK293T, 4×10⁴ cells/well for iPSCs, and 4×10⁴ cells/well for MSCs.

Hoechst staining. Cell proliferation was assessed via Hoechst staining, microscopy and counting of nuclei. Cells were incubated in KBM2 containing 2 μL Hoechst 33342 (5 mg/mL stock solution, ThermoFisher Scientific) for 30 min before imaging by epifluorescence microscopy (see details below). The number of nuclei per image was determined manually and converted in cell densities per surface area.

Immuno-fluorescence staining. Emulsions were fixed with 4% paraformaldehyde (Sigma-Aldrich, 158127) for 10 min at room temperature. Thereafter, samples were washed three times with PBS and permeabilized with 0.2% Triton X-100 (Sigma-Aldrich, ×100) for 5 min at room temperature. After washing with PBS (three times), samples were blocked for 1 h in 3% BSA. Samples were then incubated with primary antibodies at 4° C. overnight at the recommended concentration. Samples were washed six times with PBS and incubated for 1 h with the secondary antibodies, DAPI (Sigma-Aldrich, D9542, 1:1000) and Tetramethylrhodamine-phalloidin (Sigma-Aldrich, P1951, 1:500). After washing with PBS (six times), samples were transferred to μ-slide 8 wells plates (Ibidi) for imaging.

Live imaging and Image Analysis. Live imaging was performed with the Lumascope 720 housed in a CO2 incubator. Cells were incubated at 37° C. and 5% CO2 and imaged for 48 hours every 1 hour. Per well, 3 regions of interest (ROIs) were selected and 2×2 tiles were imaged. Three wells were made for each interface of interest, which brings the total ROIs to 9. After 48 hours of imaging, the tiles for each ROI were then stitched together. The colonies formed at 0 hr and after 48 hours were manually selected and the areas were measured. The stitched tiles should at least be covered with cells by 30%. Selected ROIs with less than 30% are excluded from the analysis.

Primary and Secondary Antibodies. Pluripotency markers used for immunofluorescence staining were anti-human Oct3/4 (Santa Cruz Biotechnology, SC-5279, 1:300) and anti-human Nanog (Abcam, ab109250, 1:400). The secondary antibodies used were Donkey anti-Rabbit, Alexa Fluor 647 (ThermoFisher Scientific, A-31573, 1:1000), Goat anti-Mouse, Alexa Fluor 488 (ThermoFisher Scientific, A-11029, 1:1000). For flow cytometry, antibodies used were anti-human SSEA-4 Alexa Fluor 647 (Biolegend, 330408, 1:100), anti-human TRA-1-60-R Alexa Fluor 488 (Biolegend, 330614, 1:50), anti-human Oct-4 PE (Biolegend, 653704, 1:100), and anti-human Nanog Brilliant Violet 421 (Biolegend, 674208, 1:50).

Flow Cytometry of iPSCs. Human iPSCs were cultured on TCP or βLG-SMCC-VN droplets for 4 days. Cells were washed once with PBS and dissociated from their substrate with accutase for 5 minutes at 37° C. Accutase was neutralized with E8 flex medium and centrifuged at 120×g for 3 minutes. Cells were then resuspended in E8 flex medium and counted. Surface marker staining was performed by incubating the cells in the antibody cocktail diluted in FACS buffer (PBS with 10% FBS and 0.1% NaN₃) for 30 minutes at 4° C. Cells were then washed once with FACS buffer and once with PBS by centrifuging the cells at 300×g for 5 minutes at 4° C. Cells were then incubated in Zombie Aqua Fixable dye (Biolegend, 423102, 1:500 in PBS) for 15 minutes at room temperature. Excess dye was quenched with FACS buffer and centrifuged at 300×g for 5 minutes. The cells were then fixed and permeabilized with True-Nuclear Transcription Factor buffer set (Biolegend, 424401). Fixation was done for 45 minutes and permeabilization and staining were performed according to manufacturer's instructions.

Proliferation assay CCK8. Proliferation assays were performed using the Cell Counting Kit-8 (Dojindo, Kumamoto, Japan). HEK293T growing on emulsions were incubated with 100 μl CCK-8 reagent for 4 hours at 37° C. over different time-points, and optical density at 450 nm was measured.

Proliferation assay CyQuant. Proliferation assay were performed using the CyQuant Proliferation Assay (ThermoFisher Scientific, C7026). Wells were first treated with PLL-PEG for 1 hour and washed thrice with PBS. For TCP-VN controls, wells were incubated in VN solution for 1 hour instead. βLG-SMCC-VN microdroplets of controlled sizes were then transferred into the PLL-PEG coated wells. Human iPSCs were seeded at 40,000 cells per well in a 24-well plate. The assay was performed according to manufacturer's instructions. Briefly, cells were then harvested after 1, 2, 4, and 7 days and frozen down at −80° C. After thawing the samples, cells were digested in 200 μL of CyQuant GR dye/cell lysis buffer. A ladder was generated and 1:10 dilutions of samples were plated onto an optical bottom plate (ThermoFisher Scientific; 165305). After incubation for 5 minutes in the dark, the plate was read with a microplate reader (Optima Fluostar).

Statistical analysis. Statistical analysis was carried out using OriginPro 9 through one-way ANOVA with Tukey test for posthoc analysis. Significance was determined by *P<0.05, **P<0.01, ***P<0.001 and n.s., non-significant. A full summary of statistical analysis is provided in the supplementary information.

### General

Reported herein is a range of interfaces in which, surprisingly, protein nanosheets could sustain the adhesion and expansion of adherent cells and cells (e.g. stem cells) without the use of surfactants. In all cases, it was identified that such behaviour was enabled by the strong mechanical properties and elasticity associated with corresponding nanosheet-stabilised interfaces.

The systems below were produced and tested in accordance with the materials and methods discussed above.

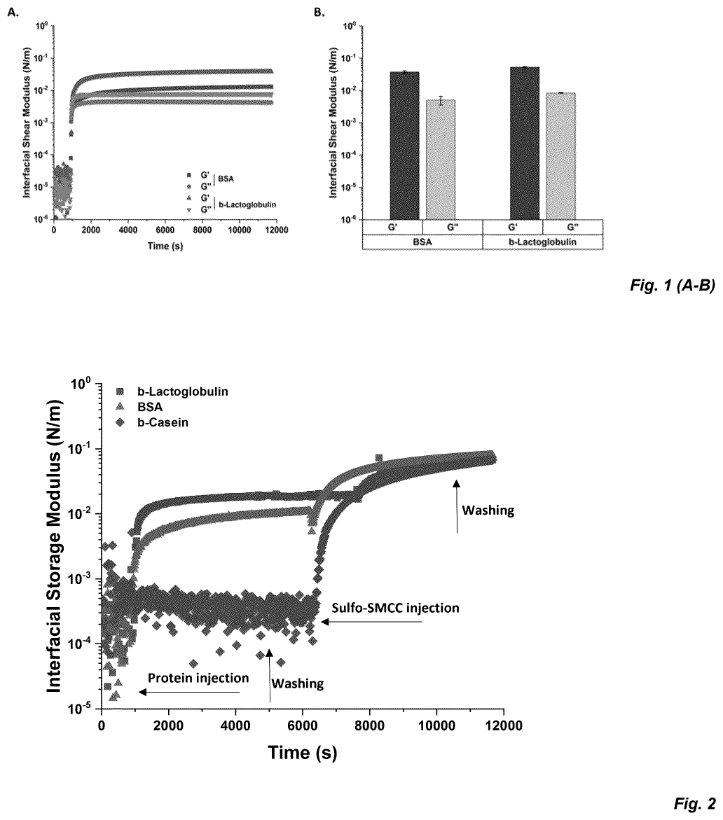

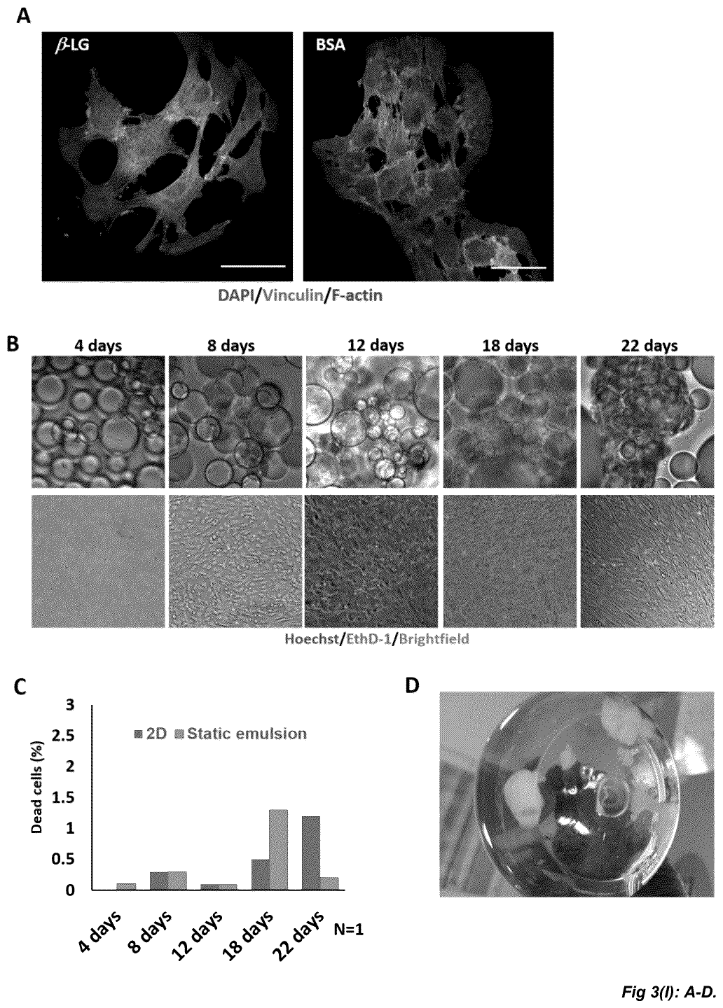

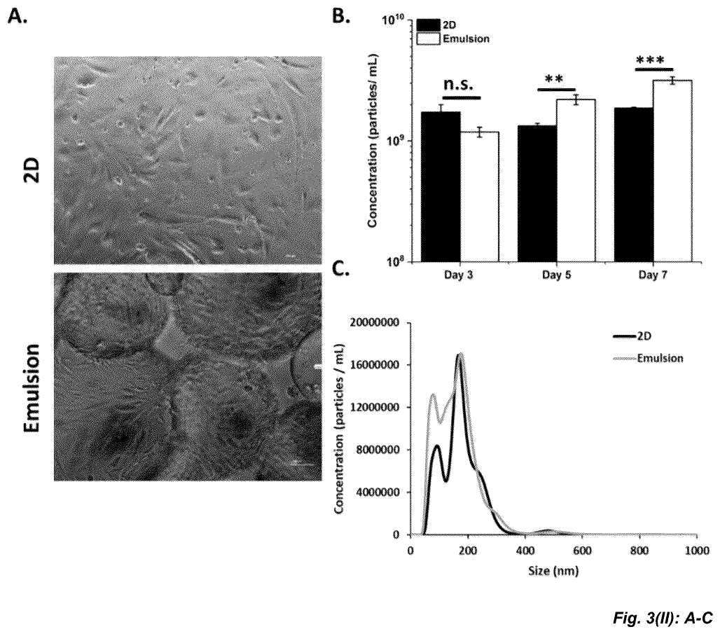

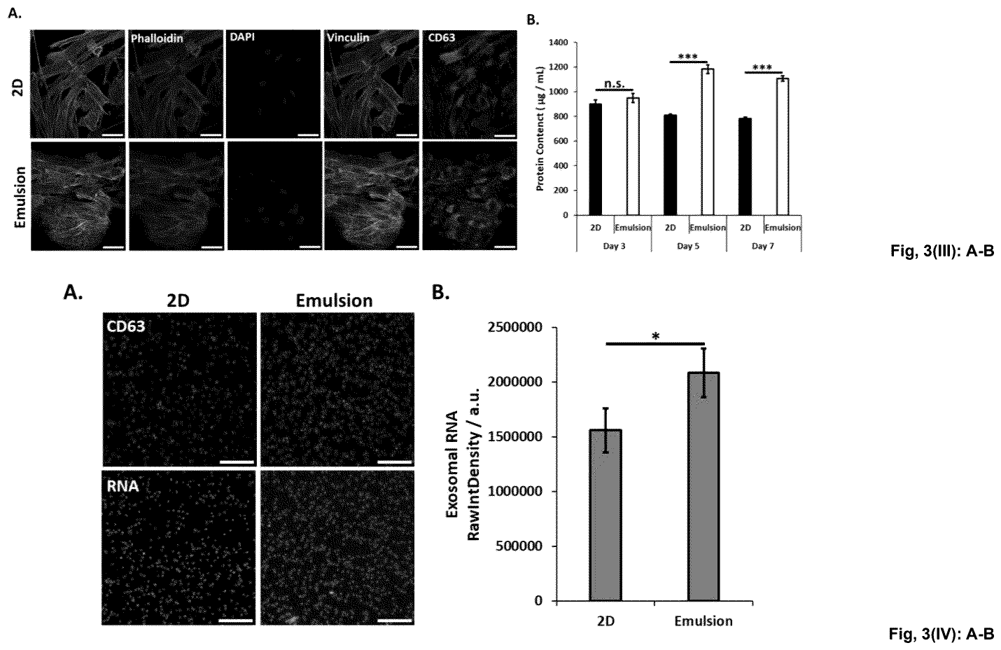

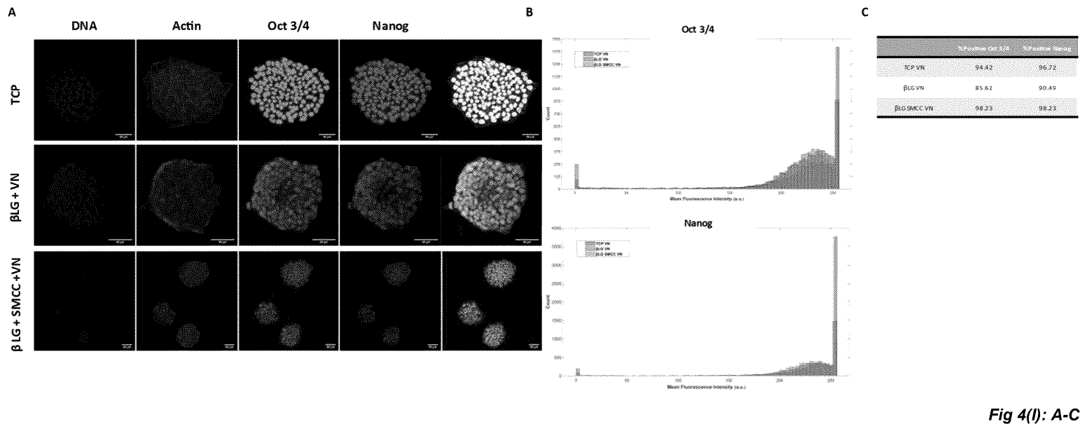

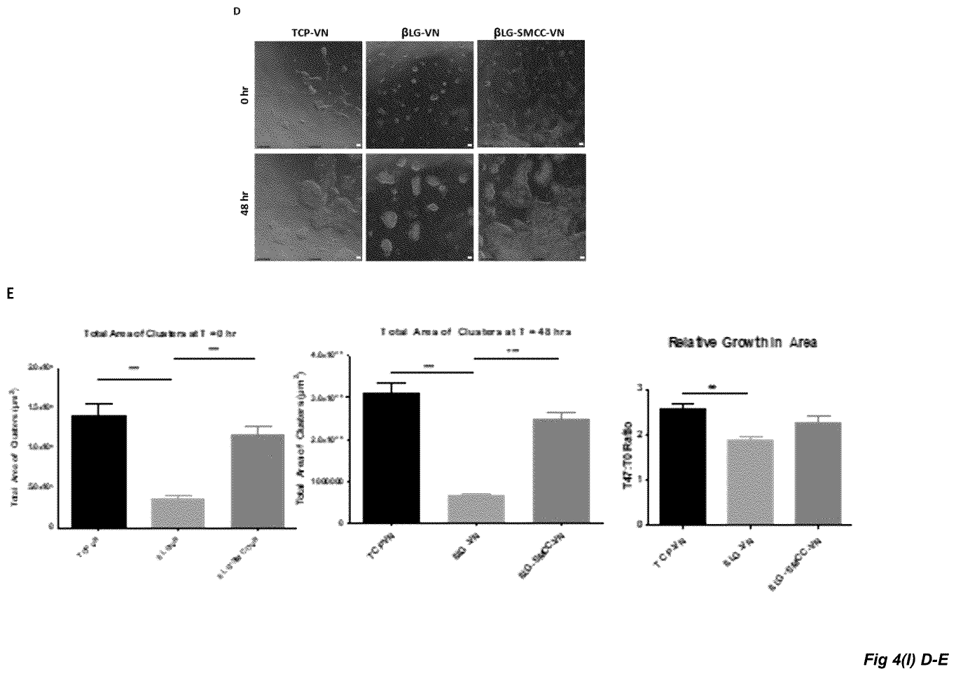

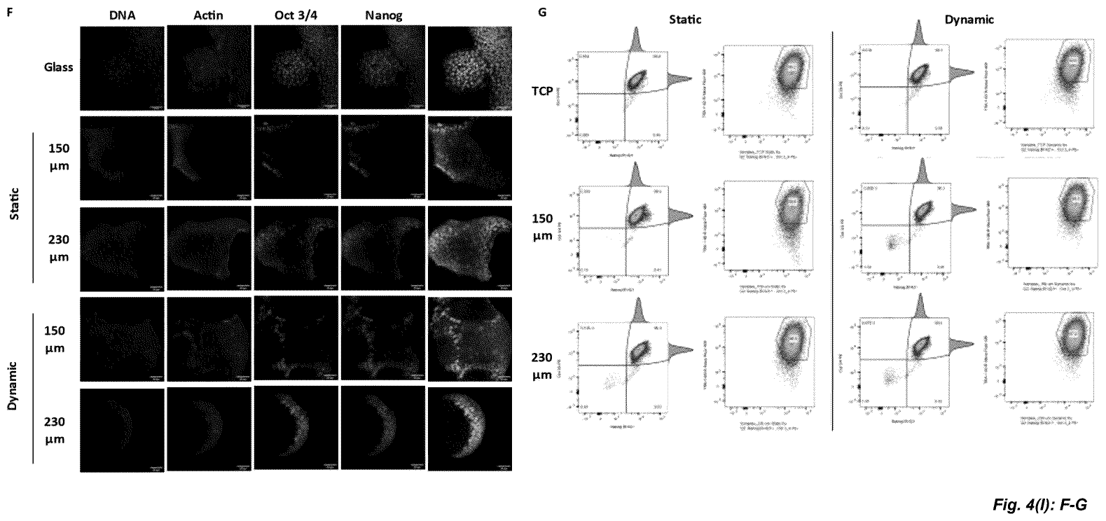

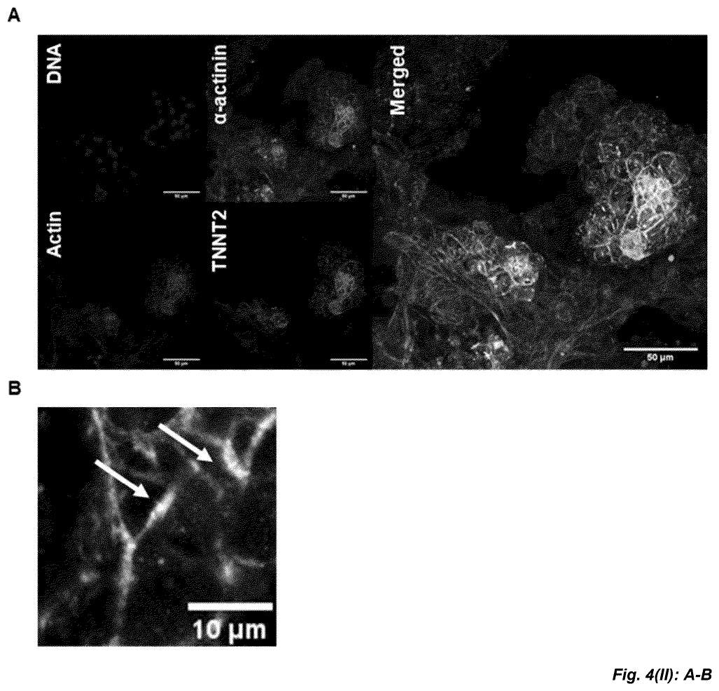

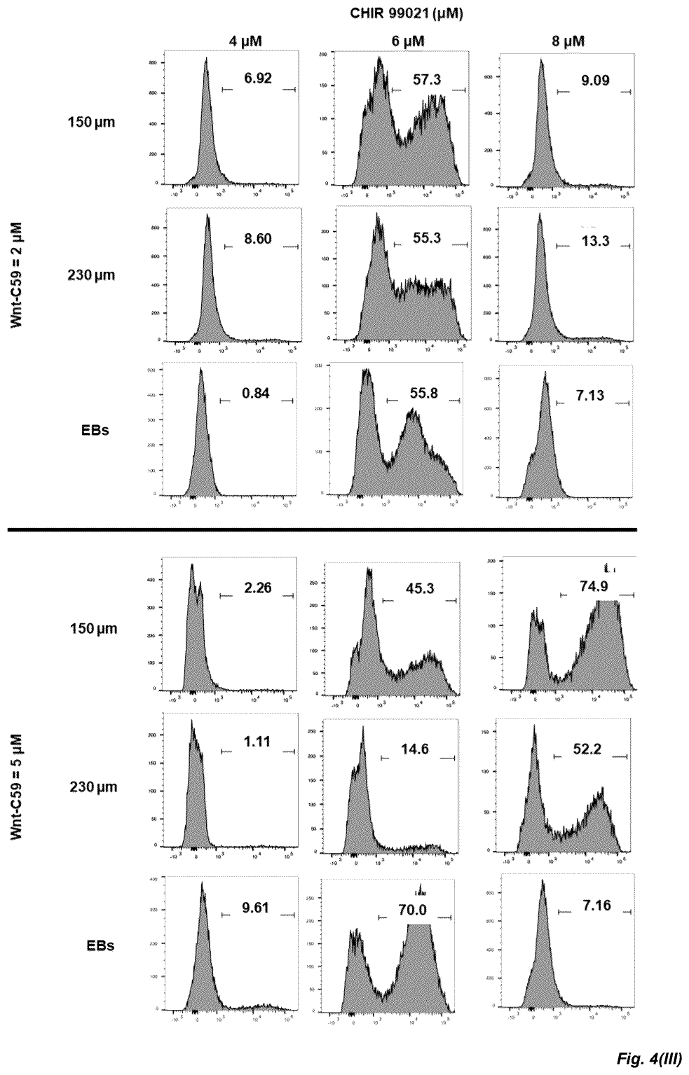

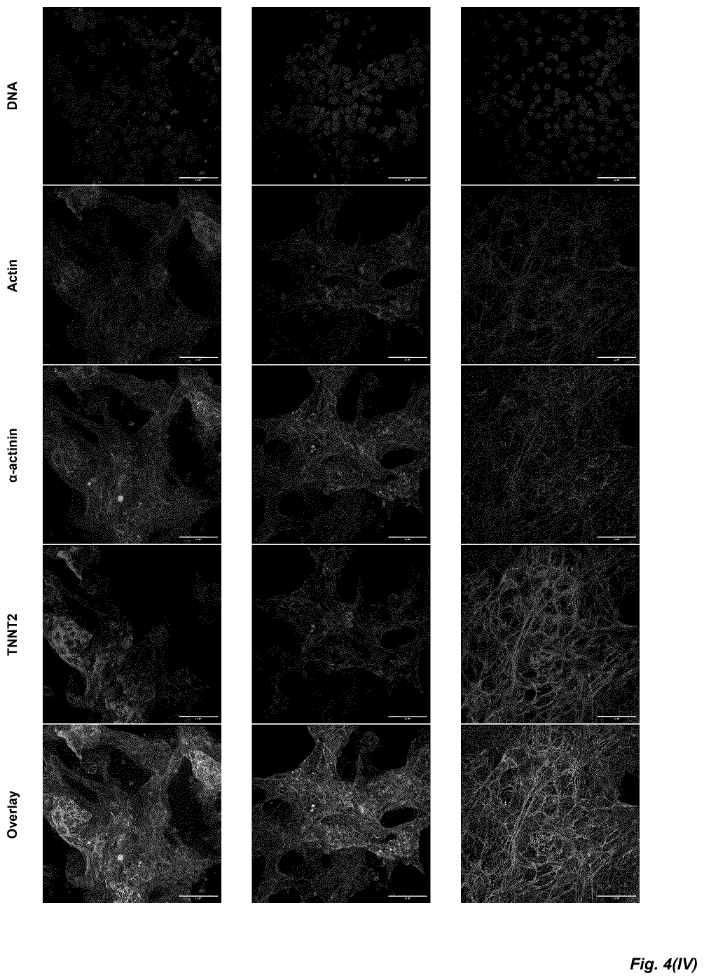

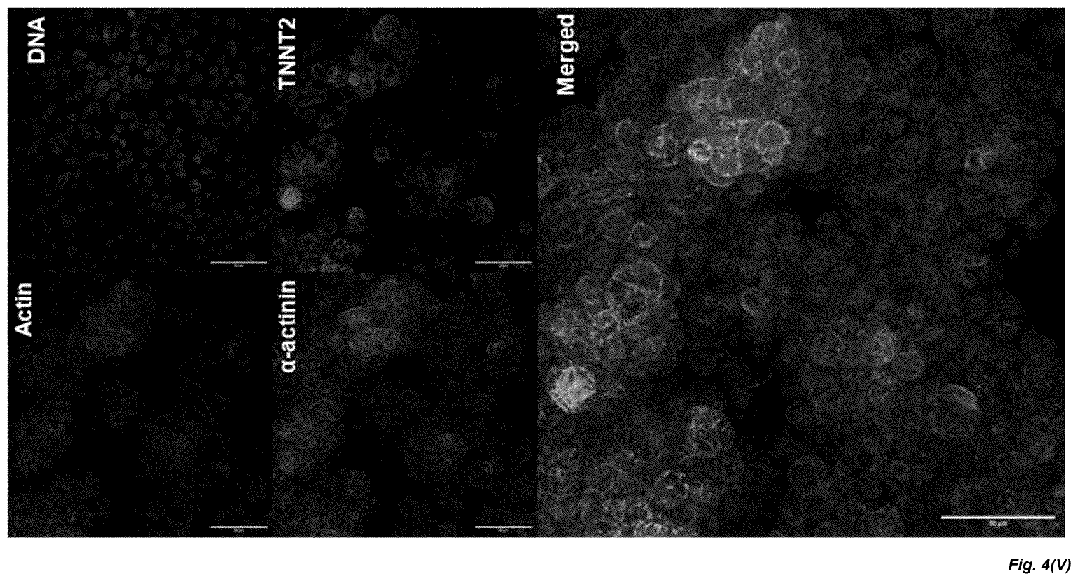

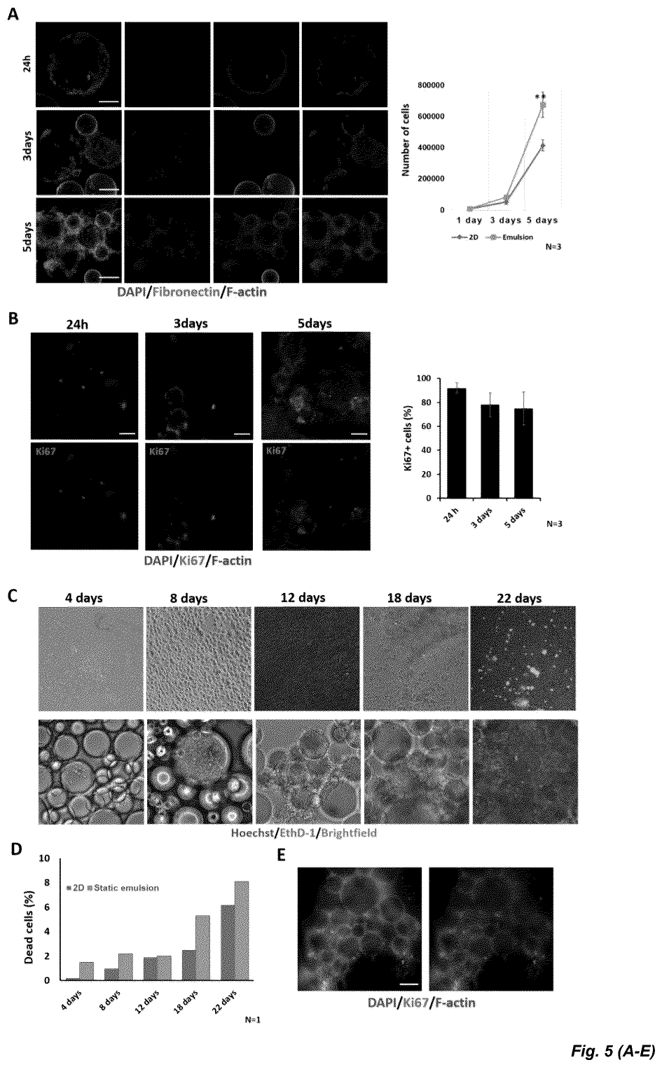

### Example 1—β-Lactoglobulin (βLG) and Bovine Serum Albumin (BSA) with Sulfo-SMCC Coupling of ECM Proteins or Peptides—See FIGS. 1 to 5

Details of specific compositions examined.
Oil phase: fluorinated oil (Novec 7500)
Protein nanosheet: 1 mg/mL BSA or 1 mg/mL βLG on PBS pH 7.4
Crosslinking of the nanosheet: 1 mg/mL Sulfosuccinimidyl 4-(N-maleimidomethyl) cyclohexane-1-carboxylate (Sulfo-SMCC) in PBS pH 7.4
Functionalisation of the interface: 1.6 mg/mL RGD peptide (CGGRGDSPG) or 20 μg/ml fibronectin in PBS pH 7.4

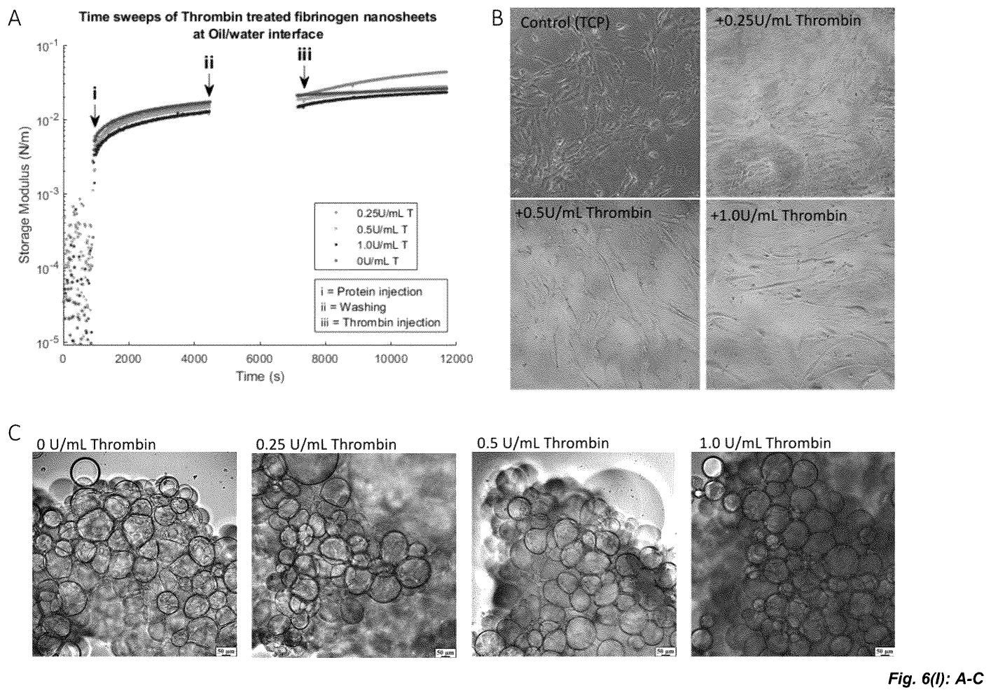

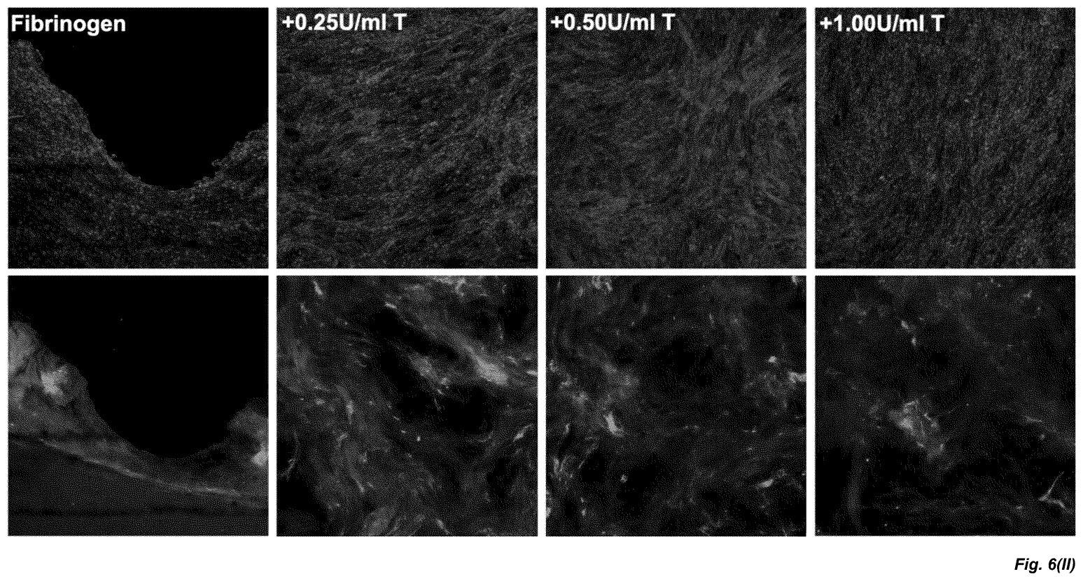

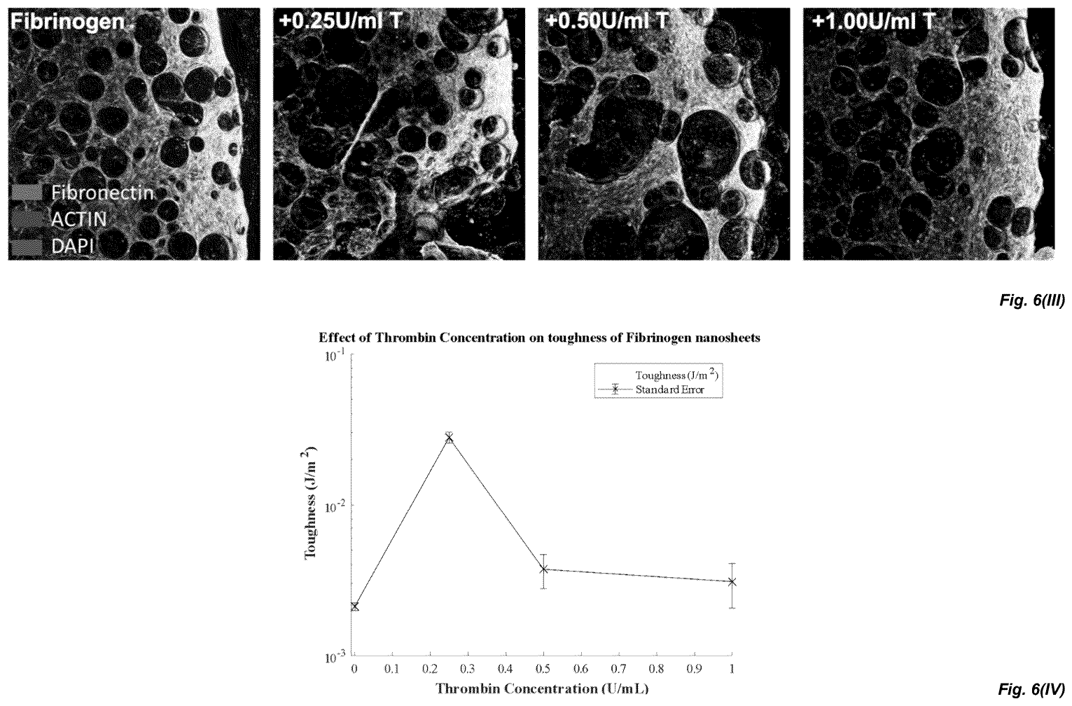

### Example 2—Fibrinogen-Stabilised Interfaces—See FIG. 6(I)

Fibrinogen is protein that appears to assemble spontaneously at liquid-liquid interfaces, forming a mechanically strong and elasticity nanosheet. Further maturation by incubation in the enzyme thrombin is also possible.

Details of specific compositions examined.
Oil phase: fluorinated oil (Novec 7500).
Protein nanosheet: 1 mg/mL fibrinogen, followed by thrombin exposure (after washing with buffer).
Crosslinking of the nanosheet: Denaturation upon adsorption and via thrombin treatment.
Functionalisation of the interface: direct adsorption of fibronectin (20 μg/mL).

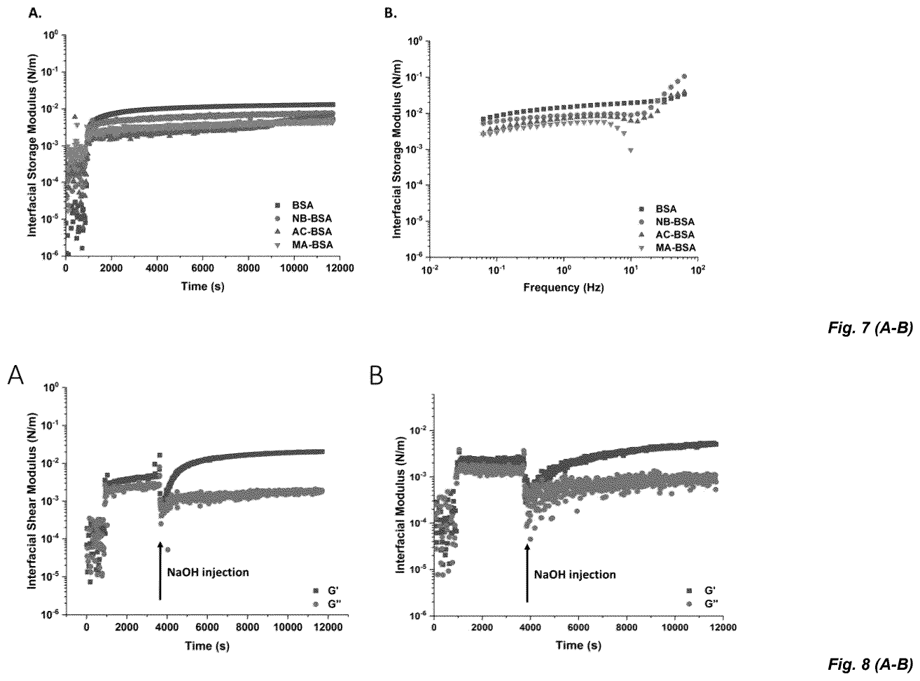

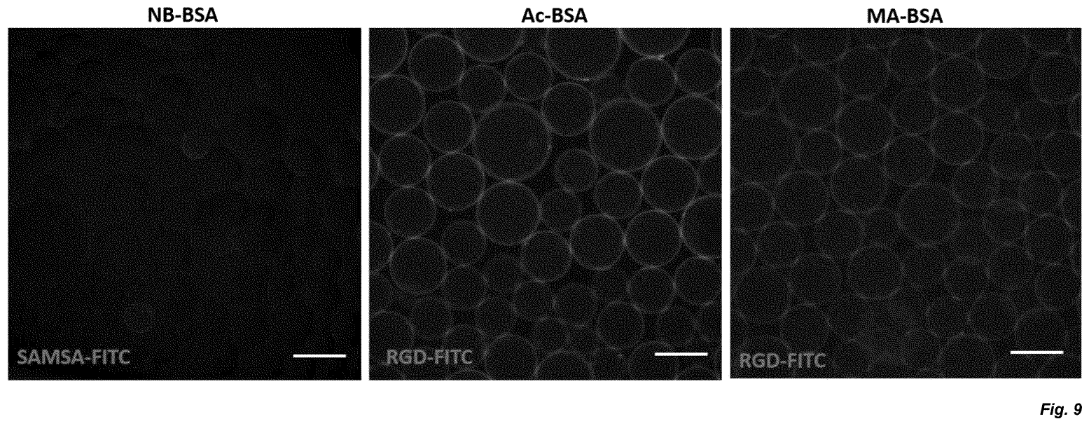

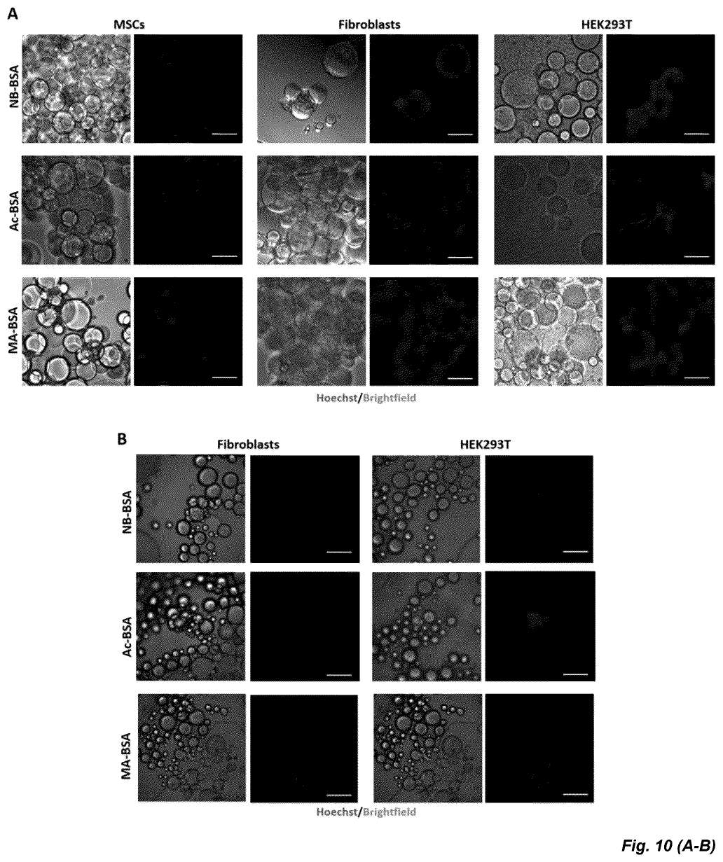

### Example 3—Interfaces Stabilised by Reactive Albumins—See FIGS. 7 to 10

Albumins are affordable, simple to engineer recombinantly, and display excellent tensio-active properties. However, they are not bioactive and do not allow the spontaneous crosslinking of interfaces (see FIG. 1). We engineered modified bovine serum albumins (BSA) displaying acrylate (Ac-BSA), methacrylate (MA-BSA) and norbornene residues (NB-BSA). These proteins were found to display excellent tensio-active properties, yet to allow biofunctionalisation with cell-adhesive peptides. We also demonstrated that simple strategies (increase in pH) enable their crosslinking.

Details of specific compositions examined.
Oil phase: fluorinated oil (Novec 7500) or rapeseed oil.
Protein nanosheet: 1 mg/mL NB-BSA, 1 mg/mL Ac-BSA, or 1 mg/mL MA-BSA at PBS pH 7.4.
Crosslinking of the nanosheet: Treatment in PBS at pH 10.5.
Functionalisation of the interface: 1.6 mg/ml RGD peptide in PBS pH 8 for NB-BSA funtionalisation and pH 9 for Ac-BSA and MA-BSA.

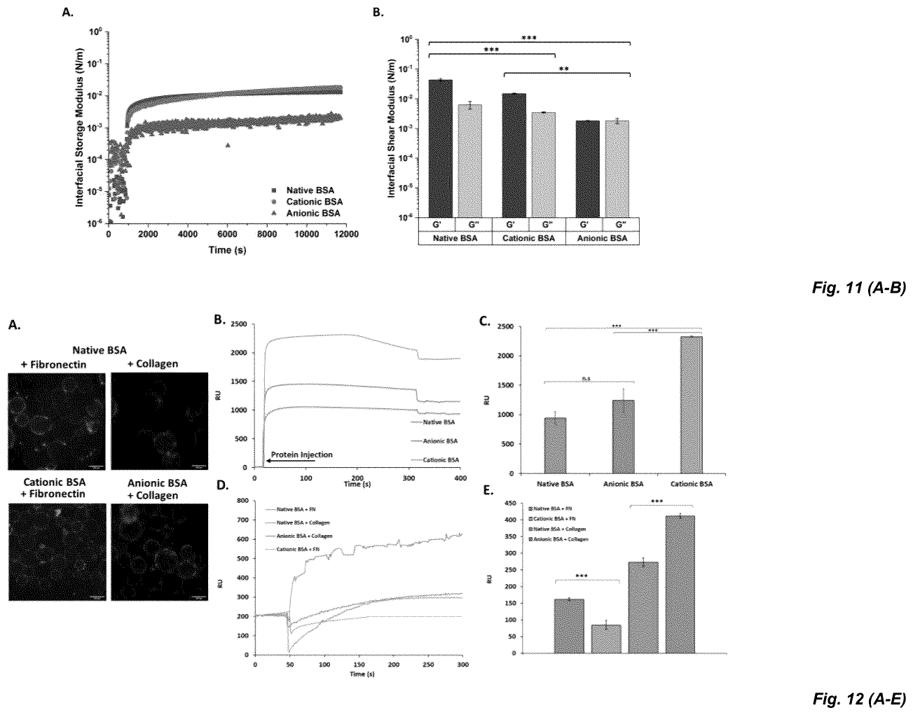

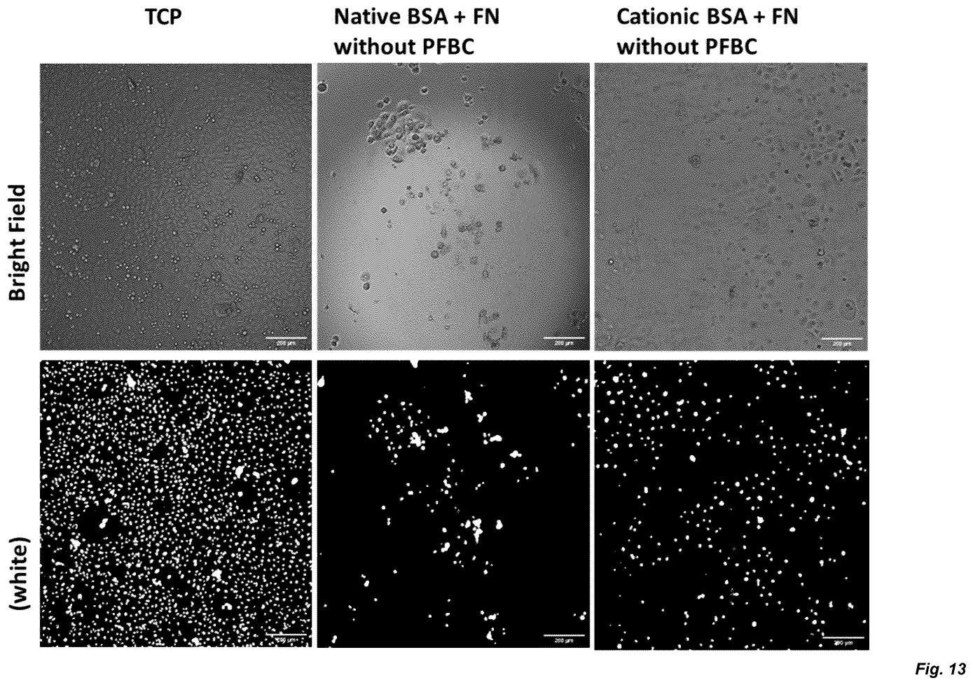

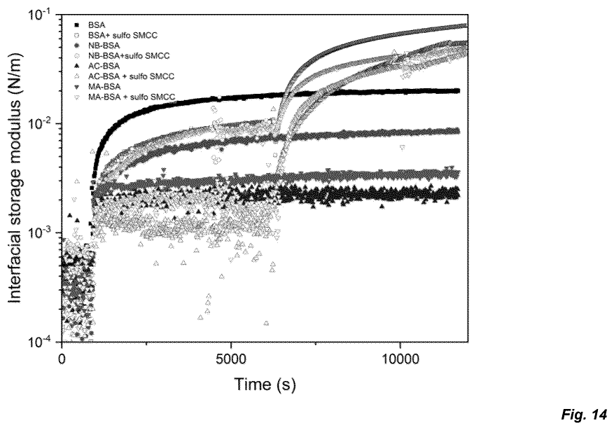

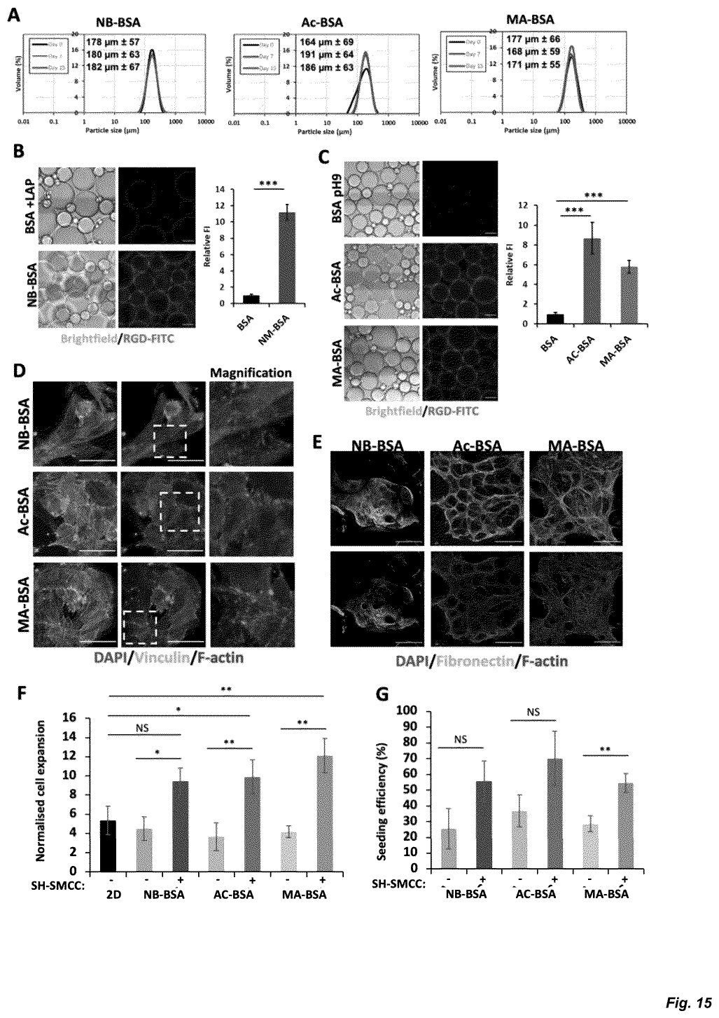

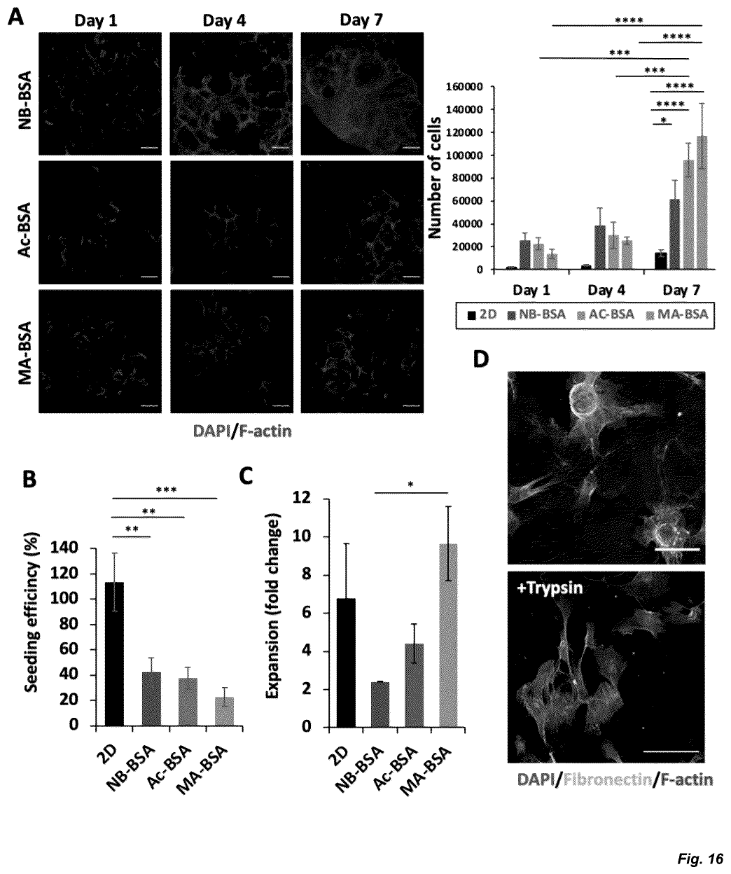

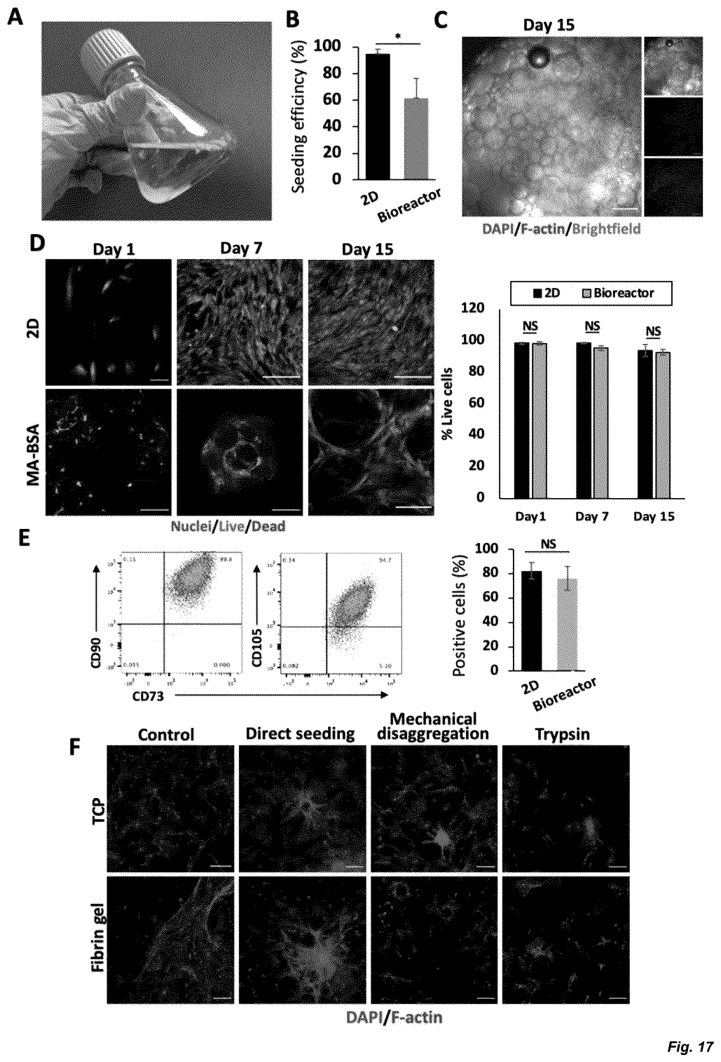

### Example 4—Keratinocytes Grown on Supercharged BSA—See FIGS. 11 to 13

Another strategy for the modification of albumin is their supercharging, or chemical modification with charged moieties. These proteins retain tensio-active properties and allow the adsorption of ECM proteins, based on electrostatic interfactions.

Details of specific compositions examined.
Oil phase: fluorinated oil (Novec 7500).
Protein nanosheet: 1 mg/mL native-BSA, 1 mg/mL cationic-BSA, or 1 mg/mL anionic-BSA on PBS pH 7.4
Coating with fibronectin or collagen: FN 20 ug/mL and collagen 100 ug/mL.

### Examples: Supplemental

The following section describes optional implementations of the examples discussed above. For instance, NB-BSA may be obtained with the following protocol:

Synthesis of functionalised BSA: methacrylated BSA (MA-BSA), acrylated BSA (AC-BSA), norbonylated BSA (NB-BSA). For NB-BSA: 10 g of native BSA (154 μmol, Sigma-Aldrich) was dissolved in 100 mL NaHCO₃/Na2CO3 buffer at pH 9.2 g of carbic anhydride (12 mmol, supplier) were slowly introduced at room temperature. The mixture was then heated under stirring at 40° C. for 4 h. A pale-yellow solution was recovered, dialysed against ultrapure water for 4 days and freeze-dried. The functionalisation degree was determined by 1H NMR by integration of the 2 alkene protons (delta ppm) of the norbornene functions normalised by the aromatic protons of BSA (delta ppm). For MA-BSA and AC-BSA, the same protocol was applied, replacing carbic anhydride with corresponding acrylate and methacrylate anhydrides.

RGD peptide coupling to biofunctionalised BSA. For the preparation of emulsions, RGD peptide coupling to functionalized BSA molecules was performed first. RGD peptide concentration was determined for each functionalized BSA formulation to enable coupling of 10 peptides per BSA molecule. Custom peptides were designed to allow reaction with functional groups in the modified BSA backbone. GCGGRGDSPG peptide was coupled to NB-BSA via Michael addition in the presence of 1% lithium phenyl-2,4,6-trimethylbenzoylphosphinate (LAP, Sigma-Aldrich), crosslinking was performed with 405 nm blue light for 3 min. at 20 mW/cm2. CGGRGDSPG peptide was conjugated to AC-BSA and MA-BSA via radical thiol-ene coupling addition in PBS pH 9 for at least 3 hours. The same procedure was used for FITC-RGD bearing peptides.

Embryoid bodies Formation. Polyvinyl alcohol (PVA) was dissolved in E8 flex medium overnight to achieve a concentration of 4% PVA. This was then filtered and supplemented with 10 μM Y-27632. iPSCs were dissociated with Gentle Cell Dissociation Reagent according to manufacturer's instructions. After centrifugation, cells were counted. An aliquot of the cell suspension is then taken and centrifuged at 120×g for 3 minutes. The cell pellet was then resuspended in E8 flex with PVA and Y-27632 to achieve a cell concentration of 1×106 cells/mL. A petri dish was then filled with sterile water. The lid was then placed upside down and multiple 10 μL drops of the cell suspension was then placed on the lid. The lid was then placed back on the petri dish resulting in hanging droplets. After 24 hours, 30 embryoid bodies were transferred to a well of an ultra-low attachment 6-well plate filled with E8 flex medium.

Cardiomyocyte Differentiation. The iPSCs were seeded on microdroplets and tissue culture plastic controls in E8 flex medium supplemented with Y-27632, a ROCK inhibitor. After 24 hours, the medium was replaced with E8 flex medium. After 4 days of culture, unless otherwise stated, cardiomyocyte differentiation was started. The cardiomyocyte differentiation protocol was adapted from Burridge et al. (2014) [165]. Briefly, a chemically defined medium composed of 3 reagents (CDM3) was used as the basal medium. It is composed of RPMI 1640 medium (w/glucose), O. sativa derived human serum albumin (has), and L-ascorbic acid 2-phosphate (LAA2P). To initiate mesoderm differentiation, the cells grown on monolayers, either on microdroplets or TCP, were incubated in CDM3 supplemented with CHIR99021 for 1 or 2 days. Afterwards, Wnt pathway was inhibited with 2 or 5 μM Wnt-C59. After 2 days, the medium was refreshed with CDM3 only and changed every other day. On the 10th day, metabolic selection was performed with CDM3L medium wherein RPMI 1640 without D-glucose was used and instead is supplemented with 5 mM sodium D-lactate. The medium was replaced with CDM3L every other day. After 6 days of metabolic selection, the medium was replenished with CDM3 afterwards.

Evaluation of emulsion stability. The stability of the different formulated emulsions was assessed at different timepoints using Mastersizer 2000 according to manufacturer's instructions. Particle size distribution was determined for each condition at the day of emulsion formation (Day 1) and after 7 days and 15 days kept at 37° C. under agitation in an orbital shaker (60 rpm). The emulsion stability was also monitored via bright-field microscopy.

Cell seeding on rapeseed oil emulsions. For cell culture on rapeseed oil emulsions, 400 μL of corresponding emulsions in medium were transferred per well in a 48 well plate treated with 25 μg/mL poly(L-lysine)-graft-polyethylene glycol (PLL-g-PEG, SuSoS). Then, excess media was removed from each well and 50 μL of MSCs at a concentration of 2×10⁶ cells/ml were seeded on top of the emulsion plug. Cells were left in the incubator for 24 h to allow attachment. The emulsion plugs were then transferred to 24 well-plates containing 50 ml medium per well.

Conical flask bioreactor scale-up. Scale up of the MA-BSA bioemulsions system was performed on 250 ml polycarbonate Erlenmeyer flasks (Corning) with Vent caps, using a total amount of medium of 50 ml. Volumes and amounts of further reagents and cells used where increased 50 times with respect to those in the culture on 24 well-plates. 5 ml of emulsions were used per flask while. Cell numbers/surface area used in bioemulsions and 2D conditions were the same (1000 cells/cm²) and calculated assuming and average diameter of 100 μm for the bioemulsions. Cells were kept under gentle agitation of 60 rpm on an orbital shaker placed inside an incubator at 37° C. in a humidified atmosphere with 5% CO₂.

Re-seeding experiments. Cells grown on emulsions were reseeded to evaluate their capacity to proliferate after growing on these conditions. MSCs grown on rapeseed oil MA-BSA emulsions for 7 days in a 24-well plate were detached using Accutase for 5 min at 37° C., reseeded on a glass coverslip and grown for 2 days. MSCs grown for 15 days on MA-BSA fluorinated oil emulsion in an Erlenmeyer flask were reseeded either on glass coverslips or on 10 mg/ml bovine fibrinogen (Sigma-Aldrich) and 2 U/mL Thrombin (Sigma-Aldrich) gels fibrinogen. Cell preparation for reseeding was done using different approaches. Cells were either (1) seeded in the gel without detachment from the emulsions, (2) gentle disaggregation by pipetting up and down with a needle, and (3) treating with Accutase for 5 min at 37° C.

Primary and Secondary Antibodies. Pluripotency markers used for immunofluorescence staining were anti-human Oct3/4 (Santa Cruz Biotechnology, SC-5279, 1:300), anti-human Nanog (Abcam, ab109250, 1:400), anti-Vinculin (#V9264, Sigma-Aldrich, 1:500) and anti-Fibronectin (#F3648, Sigma-Aldrich, 1:500). The secondary antibodies used were Donkey anti-Rabbit, Alexa Fluor 647 (ThermoFisher Scientific, A-31573, 1:1000), Goat anti-Mouse, Alexa Fluor 488 (ThermoFisher Scientific, A-11029, 1:1000). For flow cytometry, antibodies used were anti-human SSEA-4 Alexa Fluor 647 (Biolegend, 330408, 1:100), anti-human TRA-1-60-R Alexa Fluor 488 (Biolegend, 330614, 1:50), anti-human Oct-4 PE (Biolegend, 653704, 1:100), anti-human Nanog Brilliant Violet 421 (Biolegend, 674208, 1:50), FITC anti-CD73 (#MCA6068F, Bio-Rad), APC anti-CD90 (#MCA90APC, Bio-Rad), and PerCP/Cy5 anti-CD105 (#323216, BioLegend).

Flow cytometry of MSCs. Single cell suspensions of cells growing on emulsions and in 2D were obtained via Accutase treatment and agitation. Cells were then washed in 0.1% BSA and centrifuged (300×g) for 10 min at 4° C. Cells were stained for 45 minutes at RT using fluorescence-labelled antibodies and Calcein Violet (#C34858, Thermo Fisher Scientific) for cell viability. Labelled cells were then washed in PBS+1% BSA and analyzed on a FACSCanto II flow cytometer (BD Biosciences). Data analysis was performed using FlowJo software (Tree Star).

Flow Cytometry. The staining buffer was composed of 10% Foetal Bovine Serum and 0.1% NaN3. The antibodies were diluted in staining buffer. Cells were then counted and stained with the staining solution for surface markers. After 30 minutes of incubation at 4° C. in the dark, the cells were washed 3× with the staining buffer and 1× with PBS. The cells were then incubated with Zombie Aqua (1:500 dilution in PBS) for 15 minutes. The Zombie Aqua was used as a viability dye. Excess zombie aqua was quenched off with staining buffer and the cells were then fixed and permeabilized with True-Nuclear Transcription Factor Staining Kit (Biolegend) according to manufacturer's protocol. The cells were then stained for intracellular markers and incubated in the dark for 30 minutes at RT. Excess antibodies were washed off 3× with the staining buffer and the cell pellet was resuspended in 500 μL of staining buffer. Stained cells were analysed with BD LSRII. Controls used were unstained and single colour controls. The data was analysed using FlowJo software.

The disclosure also comprises the following clauses, which may be claimed:
1. A method of culturing adherent cells at a liquid-liquid interface in a cell culture system, the cell culture system comprising:
   a. an aqueous cell culture medium; and
   b. an oil or lipid phase,
   wherein the oil/lipid phase is functionalised with a conditioning layer that is disposed between the aqueous cell culture medium and the oil/lipid phase and the conditioning layer comprises a self-surface-activating amphiphilic protein or self-surface-activating amphiphilic peptide layer;
   the method comprising culturing the adherent cells in the cell culture system at the interface between the aqueous cell culture medium and the oil/lipid phase.
2. The method of clause 1, with the proviso that the conditioning layer and/or oil/lipid phase (optionally the conditioning layer, optionally the conditioning layer and oil/lipid phase) substantially do not comprise a chemical surfactant.
3. The method of clause 1, with the proviso that the conditioning layer and/or oil/lipid phase (optionally the conditioning layer, optionally the conditioning layer and oil/lipid phase) substantially do not comprise a surfactant selected from the group consisting of:
   pentafluorobenzoyl chloride (PFBC), pentadecafluorooctanoyl chloride (PDFC), octanoyl chloride, sebacoyl chloride and/or heptadecanoyl chloride;
   optionally with the proviso that the conditioning layer and/or oil/lipid phase (optionally the conditioning layer, optionally the conditioning layer and oil/lipid phase) substantially do not comprise an acyl chloride surfactant.
4. The method of clause 1, with the proviso that the conditioning layer and/or oil/lipid phase (optionally the conditioning layer, optionally the conditioning layer and oil/lipid phase) substantially do not comprise a reactive surfactant (e.g. able to form covalent bonds with the self-surface-activating amphiphilic protein or self-surface-activating amphiphilic peptide of the conditioning layer), optionally wherein the reactive surfactant has one or more reactive functional groups selected from acyl chlorides, isocyanates, maleimides, succinimide esters, azides, alkynes, alkyl halides or thiols.
5. The method of clause 1, with the proviso that the conditioning layer and/or oil/lipid phase (optionally the conditioning layer, optionally the conditioning layer and oil/lipid phase) substantially do not comprise an acyl chloride surfactant.
6. The method of any preceding clause, with the proviso that the conditioning layer and/or oil/lipid phase (optionally the conditioning layer, optionally the conditioning layer and oil/lipid phase) substantially do not comprise a small molecule chemical surfactant, such as a chemical surfactant having a molecular weight up to about 500 Da, optionally up to about 600 Da, optionally up to about 700 Da, optionally up to about 800 Da, optionally up to about 900 Da, optionally up to about 1,000 Da.
7. The method of any preceding clause, with the proviso that there is no polymer, protein or peptide layer between the conditioning layer and the oil/lipid phase.
8. The method of any preceding clause, wherein the self-surface-activating amphiphilic-protein and/or self-surface-activating amphiphilic-peptide have a molecular weight of:
   at least about 500 Da, optionally at least about 2500 Da, optionally at least about 5,000 Da, optionally at least about 7,500 Da, optionally at least about 10,000 Da, optionally at least about 20,000 Da; and/or
   at most about 10 MDa, optionally at most about 7.5 MDa, optionally at most about 5 MDa; and/or
   about 5 to 500 kDa, optionally about 10 to 350 kDa, optionally about 20 to 350 kDa.
9. The method of any preceding clause, wherein the conditioning layer consists essentially of (optionally consists of) the self-surface-activating amphiphilic protein or self-surface-activating amphiphilic peptide (e.g. wherein the method comprises washing to remove other, non-proteinaceous, components).
10. The method of any preceding clause, wherein the self-surface-activating amphiphilic protein or self-surface-activating amphiphilic peptide crosslink spontaneously at the oil/lipid-water interface.
11. The method of any one of clause 1 to 10, wherein the self-surface-activating amphiphilic protein or self-surface-activating amphiphilic peptide is crosslinked after assembly at the oil/lipid-water interface.
12. The method of any preceding clause, wherein the self-surface-activating amphiphilic protein or self-surface-activating amphiphilic peptide is:
   fibrinogen (optionally further comprising incubation in a crosslinking protease such as thrombin after assembly of the self-surface-activating amphiphilic protein or self-surface-activating amphiphilic peptide at the liquid-liquid interface); or
   modified with charged moieties (e.g. the protein/peptide is anionic or cationic such as anionic BSA, optionally anionic and in combination with a collagen layer between the conditioning layer and the aqueous cell culture medium, optionally with anionic BSA).
13. The method of any preceding clause, wherein the self-surface-activating amphiphilic protein or self-surface-activating amphiphilic peptide is modified with an agent selected from N-hydroxysuccinimide esters, pentafluorobenzoic esters, anhydrides, maleimides, acrylates, methacrylates, thiols, alkynes, azides, cyclic alkynes, haloalkanoates, isocyanates and/or epoxide.
14. The method of any preceding clause, wherein the self-surface-activating amphiphilic protein or self-surface-activating amphiphilic peptide is modified with an agent selected from N-hydroxysuccinimide esters, pentafluorobenzoic esters, maleimides, acrylates, methacrylates, thiols, alkynes, azides, cyclic alkynes, haloalkanoates, isocyanates and/or epoxide.
15. The method of any preceding clause, wherein the self-surface-activating amphiphilic protein or self-surface-activating amphiphilic peptide is modified with an agent selected from:
   1. Sulfo-SMCC (4-(N-Maleimidomethyl)cyclohexane-1-carboxylic acid 3-sulfo-N-hydroxysuccinimide ester sodium salt);
   2. Succinimidyl-trans-4-(N-maleimidylmethyl)cyclohexane-1-carboxylate;
   3. 3-(Maleimido)propionic acid N-hydroxysuccinimide ester;
   4. 6-Maleimidohexanoic acid N-hydroxysuccinimide ester;
   5. Poly(ethylene glycol) (N-hydroxysuccinimide 5-pentanoate) ether N′-(3-maleimidopropionyl)aminoethane;
   6. Poly(ethylene glycol) bis (N-hydroxysuccinimide) ester;
   7. Poly(ethylene glycol) dithiol;
   8. Poly(ethylene glycol) bismaleimide;
   9. Poly(ethylene glycol) bisacrylate;
   10. Poly(ethylene glycol) bismethacrylate;
   11. Poly(ethylene glycol) bisazide;
   12. Tetra arm and multi-arm polyethylene glycol, end functionalised with thiol,
   succinimide (e.g. succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester),
   maleimide (e.g. PEG-maleimide, PEG-bis maleimide, PEG-tetra arm maleimide or other maleimide-functionalised polymers),
   acrylate (e.g. Ac-BSA),
   methacrylate (e.g. MA-BSA),
   norbornene (e.g. NB-BSA),
   vinyl/olefin,
   tyramine,
   tyrosine,
   reactive (cross-linkable) aromatic,
   alkyne,
   strained alkyne,
   azide, or
   haloalkanoate,
   preferably wherein the one or more crosslinking moieties are selected from acrylate,
   methacrylates and norbornene
   13. Thiolated hyaluronic acid;
   14. Thiolated gelatin;
   15. Thiolated dextran;
   16. Thiolated polymers;
   17. Anhydrides, such as of palmitic acid and adipic acid, and/or mixtures of aliphatic acids and acetic anhydride (e.g. including palmitic acid, adipic acid and/or acetic anhydride); and/or
   18. Mixtures of aliphatic carboxylic acids and acetic anhydride.
16. The method of any preceding clause, wherein the self-surface-activating amphiphilic protein or self-surface-activating amphiphilic peptide is modified with an agent selected from:
   1. Sulfo-SMCC (4-(N-Maleimidomethyl)cyclohexane-1-carboxylic acid 3-sulfo-N-hydroxysuccinimide ester sodium salt);
   2. Succinimidyl-trans-4-(N-maleimidylmethyl)cyclohexane-1-carboxylate;
   3. 3-(Maleimido)propionic acid N-hydroxysuccinimide ester;
   4. 6-Maleimidohexanoic acid N-hydroxysuccinimide ester;
   5. Poly(ethylene glycol) (N-hydroxysuccinimide 5-pentanoate) ether N′-(3-maleimidopropionyl)aminoethane;
   6. Poly(ethylene glycol) bis (N-hydroxysuccinimide) ester;
   7. Poly(ethylene glycol) dithiol;
   8. Poly(ethylene glycol) bismaleimide;
   9. Poly(ethylene glycol) bisacrylate;
   10. Poly(ethylene glycol) bismethacrylate;
   11. Poly(ethylene glycol) bisazide;
   12. Tetra arm and multi-arm polyethylene glycol, end functionalised with thiol,
   succinimide (e.g. succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester),
   maleimide (e.g. PEG-maleimide, PEG-bis maleimide, PEG-tetra arm maleimide or other maleimide-functionalised polymers),
   acrylate (e.g. Ac-BSA),
   methacrylate (e.g. MA-BSA),
   norbornene (e.g. NB-BSA),
   vinyl/olefin,
   tyramine,
   tyrosine,
   reactive (cross-linkable) aromatic,
   alkyne,
   strained alkyne,
   azide, or
   haloalkanoate,
   preferably wherein the one or more crosslinking moieties are selected from acrylate,
   methacrylates and norbornene
   13. Thiolated hyaluronic acid;
   14. Thiolated gelatin;
   15. Thiolated dextran; and/or
   16. Thiolated polymers.
17. The method of any preceding clause, wherein the self-surface-activating amphiphilic protein or self-surface-activating amphiphilic peptide are functionalised prior to modification with the agent to enable reaction between the agent and modified functionality (e.g. for 7, 13, 14, 15 and/or 16 in clause 16 above), optionally wherein the self-surface-activating amphiphilic protein or self-surface-activating amphiphilic peptide are reactive, optionally wherein the self-surface-activating amphiphilic protein or self-surface-activating amphiphilic peptide is a reactive albumin.
18. The method of any preceding clause, wherein the self-surface-activating amphiphilic protein or self-surface-activating amphiphilic peptide comprises a recombinant cell adhesive peptide (optionally a genetically engineered recombinant cell adhesive peptide).
19. The method of clause 18 wherein the cell adhesive peptide comprises a peptide sequence selected from the group consisting of RGD, YIGSR, IKVAV, GFOGER and PHSRN or other sequences that can bind integrin receptors.
20. The method of any preceding clause, wherein the self-surface-activating amphiphilic protein or self-surface-activating amphiphilic peptide comprises recombinant albumin.
21. The method of any preceding clause, wherein the self-surface-activating amphiphilic protein or self-surface-activating amphiphilic peptide is crosslinked after assembly at the oil/lipid-water interface by protein/peptide denaturation (e.g. at neutral pH, or by pH shift, optionally wherein the shift is towards alkaline, such as to around pH 10 to 11, such as about pH 10.5).
22. The method of any preceding clause, wherein the self-surface-activating amphiphilic protein or self-surface-activating amphiphilic peptide is crosslinked after assembly at the oil/lipid-water interface by protein/peptide denaturation by pH shift to around pH 10 to 11, such as about pH 10.5.
23. The method of any preceding clause, wherein the self-surface-activating amphiphilic protein or self-surface-activating amphiphilic peptide is provided with one or more crosslinking moieties (optionally two, three, four or more crosslinking moieties, such as oligofunctional or polyfunctional).
24. The method of any preceding clause, wherein the one or more crosslinking moieties are selected from:
   Thiol (such as a thiolated polymer or oligomer, e.g. thiolated hyaluronic acid, thiolated carboxymethyl cellulose, thiolated dextran and/or thiolated gelatin),
   succinimide (e.g. succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester),
   maleimide (e.g. PEG-maleimide, PEG-bis maleimide, PEG-tetra arm maleimide or other maleimide-functionalised polymers),
   acrylate (e.g. Ac-BSA),
   methacrylate (e.g. MA-BSA),
   norbornene (e.g. NB-BSA),
   vinyl/olefin,
   tyramine,
   tyrosine,
   reactive (cross-linkable) aromatic,
   alkyne,
   strained alkyne,
   azide,
   halkyl alides, or
   anhydrides (e.g. the including palmitic acid and adipic acid, or mixtures of aliphatic carboxylic acid, including palmitic acid, adipic acid and/or acetic anhydride);
   preferably wherein:
   the one or more crosslinking moieties are selected from acrylate, methacrylates, norbornene and anhydrides.
25. The method of any preceding clause, wherein the one or more crosslinking moieties are selected from:
   Thiol (such as a thiolated polymer or oligomer, e.g. thiolated hyaluronic acid, thiolated carboxymethyl cellulose, thiolated dextran and/or thiolated gelatin),
   succinimide (e.g. succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester),
   maleimide (e.g. PEG-maleimide, PEG-bis maleimide, PEG-tetra arm maleimide or other maleimide-functionalised polymers),
   acrylate (e.g. Ac-BSA),
   methacrylate (e.g. MA-BSA),
   norbornene (e.g. NB-BSA),
   vinyl/olefin,
   tyramine,
   tyrosine,
   reactive (cross-linkable) aromatic,
   alkyne,
   strained alkyne,
   azide, or
   halkyl alides;
   preferably wherein the one or more crosslinking moieties are selected from acrylate, methacrylates and norbornene.
26. The method of any preceding clause, wherein the self-surface-activating amphiphilic protein or self-surface-activating amphiphilic peptide is provided with two, three, four or more crosslinking moieties, such as oligofunctional or polyfunctional.
27. The method of any preceding clause, wherein the self-surface-activating amphiphilic protein or self-surface-activating amphiphilic peptide is crosslinked after assembly at the oil/lipid-water interface with a crosslinker (optionally a crosslinker having two, three, four or more crosslinking moieties, such as an oligofunctional crosslinker or a polyfunctional crosslinker).
28. The method of any preceding clause, wherein self-surface-activating amphiphilic protein or self-surface-activating amphiphilic peptide is crosslinked with:
   a thiolated compound (such as a thiolated polymer or oligomer, e.g. thiolated hyaluronic acid, thiolated carboxymethyl cellulose, thiolated dextran and/or thiolated gelatin),
   a succinimide (e.g. succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester),
   a maleimide (e.g. PEG-maleimide, PEG-bis maleimide, PEG-tetra arm maleimide or other maleimide-functionalised polymers),
   an acrylate (e.g. with BSA),
   a methacrylate (e.g. with BSA),
   a norbornene (e.g. with BSA),
   a vinyl compound/olefin,
   tyramine,
   tyrosine,
   a reactive aromatic compound,
   alkyne,
   strained alkyne,
   azide,
   haloalkanoate; or
   anhydride (e.g. the including palmitic acid and adipic acid, or mixtures of aliphatic carboxylic acid, including palmitic acid, adipic acid and/or acetic anhydride);
   preferably wherein:
   the one or more crosslinking moieties are selected from acrylate, methacrylates, norbornene and anhydrides; or the one or more crosslinking moieties are selected from anhydrides or mixtures of aliphatic carboxylic acids and acetic anhydride.
29. The method of any preceding clause, wherein self-surface-activating amphiphilic protein or self-surface-activating amphiphilic peptide is crosslinked with:
   a thiolated compound (such as a thiolated polymer or oligomer, e.g. thiolated hyaluronic acid, thiolated carboxymethyl cellulose, thiolated dextran and/or thiolated gelatin),
   a succinimide (e.g. succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester),
   a maleimide (e.g. PEG-maleimide, PEG-bis maleimide, PEG-tetra arm maleimide or other maleimide-functionalised polymers),
   an acrylate (e.g. with BSA),
   a methacrylate (e.g. with BSA),
   a norbornene (e.g. with BSA),
   a vinyl compound/olefin,
   tyramine,
   tyrosine,
   a reactive aromatic compound,
   alkyne,
   strained alkyne,
   azide, or
   haloalkanoate;
   preferably wherein the one or more crosslinking moieties are selected from acrylate, methacrylates and norbornene.
30. The method of any one of clauses 25 to 29, wherein the ahydride is an anhydride of an aliphatic carboxylic acids, including palmitic acid and adipic acid, or mixtures of aliphatic carboxylic acid, including palmitic acid and adipic acid and acetic anhydride.
31. The method of any one of clauses 25 to 29, wherein the succinimide is selected from succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), N-hydroxysulfosuccinimide (sulfo-NHS), NHS ester, sulfo-NHS ester.
32. The method of any one of clauses 25 to 29, wherein the maleimide is selected from succinimidyl 4-(N-maleimidomethyl)cyclohexane-1-carboxylate (SMCC), sulfosuccinimidyl 4-(N-maleimidomethyl) cyclohexane-1-carboxylate (sulfo-SMCC) N-hydroxysuccinimide (NHS), PEG-bis maleimide, PEG-tetra arm maleimide or other maleimide-functionalised polymers.
33. The method of any one of clauses 21 to 32, wherein the self-surface-activating amphiphilic protein or self-surface-activating amphiphilic peptide is crosslinked with crosslinks provided by covalent or supramolecular bonds (hydrogen bonding, host-guest interactions, electrostatic interactions or pi-pi stacking), optionally wherein the self-surface-activating amphiphilic protein or self-surface-activating amphiphilic peptide is crosslinked with crosslinks comprising ester, amide, urethane, carbonate, ether, amine, thioether, and/or thioester moieties.
34. The method of any one of clauses 21 to 33, wherein the conditioning layer consists essentially of (optionally consists of) the self-surface-activating amphiphilic protein or self-surface-activating amphiphilic peptide and cross linkers (e.g. wherein the method comprises washing to remove other, non-cross-linked, components).
35. The method of any preceding clause, wherein the self-surface-activating amphiphilic-protein and/or self-surface-activating amphiphilic-peptide are selected from the group consisting of albumins (e.g. bovine serum albumin, BSA; human serum albumin, HSA; plant based albumin including sunflower albumin and rice albumin; lactalbumin), caseins (e.g. α-casein or β-casein), lactoglobulins (e.g. β-lactoglobulin, βLG), fibrinogen, gelatine, collagen, fibronectin, laminin, vitronectin, proteins contained in Matrigel, Geltrex or comparable cell adhesive protein formulations, hydrophobins, lysozymes, zein, heliantinin (11S), pea proteins, soy proteins, other plant-based globulins, and combinations thereof, optionally from albumins, lactoglobulins and fibrinogen, and combinations thereof.
36. The method of any preceding clause, wherein the self-surface-activating amphiphilic-protein and/or self-surface-activating amphiphilic-peptide are selected from the group consisting of albumins (e.g. bovine serum albumin, BSA; human serum albumin, HSA; plant based albumin including sunflower albumin and rice albumin; lactalbumin), caseins (e.g. α-casein or β-casein), lactoglobulins (e.g. β-lactoglobulin, βLG), fibrinogen, gelatine, hydrophobins, lysozymes, zein, heliantinin (11 S), and combinations thereof, optionally from albumins, lactoglobulins and fibrinogen, and combinations thereof.
37. The method of any preceding clause, wherein the self-surface-activating amphiphilic protein and/or self-surface-activating amphiphilic peptide are provided at a concentration of:
   at most about 500 mg/mL, optionally at most 200 mg/mL, optionally at most about 100 mg/mL; and/or
   at least about 10 ng/mL, optionally at least about 0.0001 mg/mL, optionally at least about 0.00075 mg/mL, optionally at least 0.001 mg/mL; and/or
   about 10 ng/mL to 500 mg/mL, optionally about 100 ng/mL to 200 mg/mL, optionally about 100 ng/mL to 100 mg/mL, optionally about 1 mg/mL to 100 mg/mL;
   the concentration being based on the amount of protein/peptide in the aqueous cell culture medium when they are deposited.
38. The method of any preceding clause, wherein the cell culture system is washed (e.g. with an aqueous wash such as deionised water, phosphate buffer saline or any other buffer or relevant electrolyte solution) after assembly of the interface and wherein the self-surface-activating amphiphilic protein and/or self-surface-activating amphiphilic peptide are provided at a concentration of:
   at most about 10 mg/cm², optionally at most 5 mg/cm², optionally at most about 1 mg/cm²; and/or
   at least about 1 ng/cm², optionally at least about 10 ng/cm², optionally at least about 100 ng/cm², optionally at least 1 μg/cm²; and/or
   about 10 ng/cm² to 50 μg/cm², optionally about 20 ng/cm² to 20 μg/cm², optionally about 50 ng/cm² to 10 μg/cm², optionally about 100 ng/cm² to 5 μg/cm²;
   the concentration being based on the amount of protein/peptide in the aqueous cell culture medium after washing.
39. The method of any preceding clause, wherein the conditioning layer further comprises a polysaccharide (such as hyaluronic acid or pectin), wherein the polysaccharide is cross-linked with the self-surface-activating amphiphilic protein and/or self-surface-activating amphiphilic peptide.
40. The method of clause 39, wherein the polysaccharide is hyaluronic acid and the self-surface-activating amphiphilic protein and/or self-surface-activating amphiphilic peptide is albumin, or wherein the polysaccharide is pectin and the self-surface-activating amphiphilic protein and/or self-surface-activating amphiphilic peptide is lysozyme.
41. The method of clause 40, wherein crosslinks are formed according to any one of clauses 21 to 33 (e.g. by denaturation, and/or after assembly, and/or using the same crosslinker moieties, and/or using the same crosslinkers, and/or using the same crosslinks/interactions, mutatis mutandis).
42. The method of any one of clauses 39 to 41, wherein the conditioning layer consists essentially of (optionally consists of) the self-surface-activating amphiphilic protein or self-surface-activating amphiphilic peptide, polysaccharides and cross linkers (e.g. wherein the method comprises washing to remove other, non-cross-linked, components).
43. The method of any preceding clause, wherein:
   (a) the adherent cells are stem cells; and/or
   (b) the method is for the long-term culture of adherent cells; and/or
   (c) the adherent cells are cultured for at least about 1 day, or at least about 5 days, or at least about 7 days, or at least about 14 days, or at least about 25 days; and/or
   (d) the adherent cells are cultured for at least about 7 days.
44. The method of any preceding clause, wherein at least 80% of the cells are alive at the end of the culture period.
45. The method of any preceding clause, wherein the elasticity of the liquid-liquid interface is sufficient for the cells (e.g. stem cells) to proliferate to at least about 50% confluency.
46. The method of any preceding clause, wherein the elasticity of the interface (% of stress retained at the interface in stress relaxation experiments) is between about 30% and about 100%, or about 40% and about 100%, or about 50% and about 100%, or about 60% and about 100%, or about 65% and about 90%, or about 65% and about 80%.
47. The method of any preceding clause, wherein the elasticity of the interface is at least about 30%.
48. The method of any preceding clause, wherein the elasticity of the interface (% of stress retained at the interface in stress relaxation experiments) is between about 60% and about 100%, or about 65% and about 90%, or about 65% and about 80%.
49. The method of any preceding clause, wherein the elasticity of the interface is at least about 60%,
50. The method of any preceding clause, wherein the dry thickness of the conditioning layer is from about 1 nm to about 10 μm, from about 1 nm to about 100 nm, from about 1 nm to about 50 nm and from about 1 nm to about 20 nm.
51. The method of any preceding clause, wherein the oil is selected from the group consisting of silicone oil, fluorinated oil, hydrocarbons, paraffin oil, mineral oil, fatty acid oils, or a plant-based oil optionally selected from peanut oil, sunflower oil, flax seed oil, soybean oil, castor oil, palm oil, rapeseed oil and olive oil.
52. The method of clause 51, wherein the oil is a silicone oil.
53. The method of clause 52, wherein the silicone oil is polydimethylsiloxane (PDMS) or an associated derivative.
54. The method of clause 53, wherein the oil is a fluorinated oil, optionally wherein the fluorinated oil comprises a perfluorinated aliphatic group (optionally an alkane, such as a branched alkane) and/or an alkoxy group, optionally wherein the fluorinated oil is an alkoxyperfluoroalkane.
55. The method of clause 54, wherein the fluorinated oil is an alkoxyperfluoroalkane.
56. The method of clause 54, wherein the fluorinated oil is 3-ethoxyperfluoro(2-methylhexane), Novec 7500; hexane,3-ethoxy-1,1,1,2,3,4,4,5,5,6,6,6-dodecafluoro-2-(trifluoromethyl) or 2-(trifluoromethyl)-3-ethoxydodecafluorohexane, FC-40 or perfluorodecalin.
57. The method of clause 51, wherein the oil is a mineral oil.
58. The method of clause 51, wherein the oil is plant oil, optionally rapeseed oil, peanut oil, sunflower oil, flax seed oil, soybean oil.
59. The method of any one of clauses 1 to 50, wherein the lipid is selected from saturated fatty acids, monounsaturated fatty acids, polyunsaturated fatty acids, palmitic acid, lauric acid, myristic acid, stearic acid, oleic acid, eicosenoic acid, docosahexaenoic acid, linoleic acid, or a corresponding ester thereof.
60. The method of any preceding clause, further comprising a protein or peptide layer between the conditioning layer and the aqueous cell culture medium.
61. The method of clause 60 wherein the protein or peptide layer between the conditioning layer and the aqueous cell culture medium comprises an extra-cellular matrix (ECM) protein or macromolecule mimicking the cell adhesive properties of ECM proteins.
62. The method of clause 61 wherein the extra-cellular matrix (ECM) protein or macromolecule mimicking the cell adhesive properties of ECM proteins is selected from fibronectin, vitronectin, collagen, laminin, agrin, fibroin and elastin.
63. The method of clause 61 or 62, wherein the protein or macromolecule mimicking the cell adhesive properties of ECM proteins comprises a cell adhesive peptide.
64. The method of clause 63 wherein the cell adhesive peptide sequence comprises a peptide sequence selected from the group consisting of RGD, YIGSR, IKVAV, GFOGER and PHSRN.
65. The method of any one of clauses 60 to 64, wherein the protein or peptide layer between the conditioning layer and the aqueous cell culture medium is provided at a concentration of:
   at most about 500 mg/mL, optionally at most 200 mg/mL, optionally at most about 100 mg/mL; and/or
   at least about 10 ng/mL, optionally at least about 0.0001 mg/mL, optionally at least about 0.00075 mg/mL, optionally at least 0.001 mg/mL; and/or
   about 10 ng/mL to 500 mg/mL, optionally about 100 ng/mL to 200 mg/mL, optionally about 100 ng/mL to 100 mg/mL, optionally about 1 mg/mL to 100 mg/mL;
   the concentration being based on an amount of protein or peptide added to the aqueous cell culture medium to form the layer between the conditioning layer and the aqueous cell culture medium.
66. The method of any preceding clause, wherein the self-surface-activating amphiphilic protein or self-surface-activating amphiphilic peptide spontaneously assemble(s) at the liquid-liquid interface.
67. The method of any preceding clause, further comprising incubation in a protease such as thrombin after assembly of the self-surface-activating amphiphilic protein or self-surface-activating amphiphilic peptide at the liquid-liquid interface.
68. The method of clause 67, wherein the protease is provided at a concentration of at least about 0.1 units/mL, optionally at least about 0.25 units/mL, optionally at least about 0.5 units/mL.
69. The method of any preceding clause, wherein the cells are engineered cells (e.g. to express protein, ECM proteins, antibodies, enzymes, hormones, growth factors, exosomes, viral particles, etc.) and/or are selected from a group consisting of human primary keratinocytes (HPKs), mesenchymal stem cells (MSCs), induced pluripotent stem cells (iPSCs), Chinese hamster ovary (CHO) cells, human umbilical vein endothelial cells (HUVECs), adipose derived stem cells, amniotic fluid derived stem cells, hepatocytes, lung epithelial cells, cord blood stem cells, fibroblasts, HEK293 cells, cardiomyocytes and skeletal muscle cells.
70. The method of clause 69, wherein the cells are human primary keratinocytes.
71. The method of clause 69, wherein the cells are mesenchymal stem cells.
72. The method of clause 69, wherein the cells are induced pluripotent stem cells.
73. The method of any preceding clause, wherein the cell culture system is in the form of an emulsion or in the form of a planar sheet, optionally an emulsion.
74. The method of any preceding clause, wherein the shear interfacial modulus of the interface between the aqueous medium and the functionalised oil/lipid phase is at least about 0.001 N/m, optionally at least about 0.01 N/m.
75. The method of any preceding clause, wherein the pH of the aqueous medium during formation of the conditioning layer is from about 3 to about 13, or from about 4 to about 12, or from about 5 to about 12, or from about 6 to about 11.
76. The method of any preceding clause, wherein the elasticity of the cell conditioning layer is at least about 30% (e.g. at least about 60%), the shear interfacial modulus of the interface between the aqueous medium and the oil/lipid phase is at least about 0.001 N/m, optionally at least about 0.01 N/m, and the pH of the cell conditioning layer is from about 6 to about 11.
77. The method of any preceding clause, further comprising seeding the cells at the liquid-liquid interface of the cell culture system.
78. The method of any preceding clause, further comprising harvesting the cultured cells from the cell culture system.
79. The method of clause 78, wherein harvesting the cells does not involve enzymatic digestion or temperature treatment.
80. A culture system for the culture of adherent cells at a liquid-liquid interface, the culture system comprising:
   a. an aqueous cell culture medium; and
   b. an oil or lipid phase, wherein the oil/lipid phase is functionalised with a conditioning layer that is disposed between the aqueous cell culture medium and the oil/lipid phase and the conditioning layer comprises a self-surface-activating amphiphilic protein or self-surface-activating amphiphilic peptide layer;
   the system comprising culturing the adherent cells at the interface between the oil/lipid phase and the aqueous cell culture medium.
81. The culture system of clause 80, as further defined according to the features of any of clauses 1 to 79.
82. Use of the culture system defined in clause 80 or clause 81 for the culture of adherent cells at a liquid-liquid interface.
83. A method of expanding a population of adherent cells comprising culturing the cells at a liquid-liquid interface according to the method of any one of clauses 1 to 79 and harvesting the cells from the culture medium.
84. The method according to clause 83 wherein the adherent cells are cultured to at least 30% confluence prior to harvesting.
85. A bioreactor comprising a culture of adherent cells (optionally stem cells), wherein the cells adhere to a liquid-liquid interface in a cell culture medium defined in clause 80 or 81.
86. The bioreactor of clause 85, wherein the cells (e.g. stem cells) are at 30% confluence or above.
87. The bioreactor of clause 85 or clause 86, wherein the bioreactor is a cell culture flask or bag.
88. A kit of parts comprising combinations of one or more oils or lipids, and one or more self-surface-activating amphiphilic proteins or self-surface-activating peptides for the culture of adherent cell and optionally stem cells, optionally wherein the oil/lipid(s) and self-surface-activating amphiphilic protein(s)/self-surface-activating amphiphilic peptide(s) are as defined in any one of clauses 1 to 79.

Any listing or discussion of an apparently prior-published document in this specification should not necessarily be taken as an acknowledgement that the document is part of the state of the art or common general knowledge. All references disclosed herein are to be considered to be incorporated herein by reference.

Those skilled in the art will recognise or be able to ascertain using no more than routine experimentation many equivalents to the specific implementations described herein. The scope of the present disclosure herein is not intended to be limited to the above description, but rather is as set forth in the appended claims. Those of ordinary skill in the art will appreciate that various changes and modifications to this description may be made without departing from the spirit or scope of the present disclosure.

1. A method of culturing adherent cells at a liquid-liquid interface in a cell culture system, the cell culture system comprising:
a. an aqueous cell culture medium; and
b. an oil or lipid phase,
wherein the oil/lipid phase is functionalised with a conditioning layer that is disposed between the aqueous cell culture medium and the oil/lipid phase and the conditioning layer comprises a self-surface-activating amphiphilic protein or self-surface-activating amphiphilic peptide layer;
the method comprising culturing the adherent cells in the cell culture system at the interface between the aqueous cell culture medium and the oil/lipid phase,
with the proviso that the conditioning layer and/or oil/lipid phase substantially do not comprise a chemical surfactant.

2. The method of claim 1, with the proviso that the conditioning layer and/or oil/lipid phase substantially do not comprise a surfactant selected from the group consisting of:
pentafluorobenzoyl chloride (PFBC), pentadecafluorooctanoyl chloride (PDFC), octanoyl chloride, sebacoyl chloride and/or heptadecanoyl chloride.

3. The method of claim 1, with the proviso that the conditioning layer and/or oil/lipid phase substantially do not comprise a reactive surfactant.

4. The method of claim 1, with the proviso that the conditioning layer and/or oil/lipid phase substantially do not comprise an acyl chloride surfactant.

5. The method of claim 1, with the proviso that the conditioning layer and/or oil/lipid phase substantially do not comprise a small molecule chemical surfactant, such as a chemical surfactant having a molecular weight up to about 500 Da.

6. The method of claim 1, with the proviso that there is no polymer, protein or peptide layer between the conditioning layer and the oil/lipid phase.

7. The method of claim 1, wherein the self-surface-activating amphiphilic-protein and/or self-surface-activating amphiphilic-peptide have a molecular weight of
about 5 to 500 kDa.

8. The method of claim 1, wherein the conditioning layer consists essentially of the self-surface-activating amphiphilic protein or self-surface-activating amphiphilic peptide.

9. The method of claim 1, wherein the self-surface-activating amphiphilic protein or self-surface-activating amphiphilic peptide crosslink spontaneously at the oil/lipid-water interface.

10. The method of claim 1, wherein the self-surface-activating amphiphilic protein or self-surface-activating amphiphilic peptide is crosslinked after assembly at the oil/lipid-water interface.

11. The method of claim 1, wherein the self-surface-activating amphiphilic protein or self-surface-activating amphiphilic peptide is
fibrinogen.

12. The method of claim 1, wherein the self-surface-activating amphiphilic protein or self-surface-activating amphiphilic peptide are functionalised prior to modification with the agent to enable reaction between the agent and modified functionality.

13. The method of claim 1, wherein the self-surface-activating amphiphilic protein or self-surface-activating amphiphilic peptide comprises a recombinant cell adhesive peptide.

14. The method of claim 1, wherein the self-surface-activating amphiphilic protein or self-surface-activating amphiphilic peptide is crosslinked after assembly at the oil/lipid-water interface by protein/peptide denaturation.

15. The method of claim 1, wherein the self-surface-activating amphiphilic protein or self-surface-activating amphiphilic peptide is provided with one or more crosslinking moieties.

16. The method of claim 1, wherein the one or more crosslinking moieties are selected from acrylate, methacrylates, norbornene and anhydride.

17. The method of claim 14, wherein the conditioning layer consists essentially of the self-surface-activating amphiphilic protein or self-surface-activating amphiphilic peptide and cross linkers.

18. The method of claim 1, wherein the cell culture system is washed after assembly of the interface and wherein the self-surface-activating amphiphilic protein and/or self-surface-activating amphiphilic peptide are provided at a concentration of:
about 10 ng/cm² to 50 μg/cm²;
the concentration being based on the amount of protein/peptide in the aqueous cell culture medium after washing.

19. The method of claim 1, wherein the conditioning layer further comprises a polysaccharide, wherein the polysaccharide is cross-linked with the self-surface-activating amphiphilic protein and/or self-surface-activating amphiphilic peptide.

20. The method of claim 19, wherein the conditioning layer consists essentially of the self-surface-activating amphiphilic protein or self-surface-activating amphiphilic peptide, polysaccharides and cross linkers.

21. The method of claim 1, wherein the protein or peptide layer between the conditioning layer and the aqueous cell culture medium comprises an extra-cellular matrix (ECM) protein or macromolecule mimicking the cell adhesive properties of ECM proteins, and wherein the extra-cellular matrix (ECM) protein or macromolecule mimicking the cell adhesive properties of ECM proteins is selected from fibronectin, vitronectin, collagen, laminin, agrin, fibroin and elastin.

22. The method of claim 1, wherein the protein or peptide layer between the conditioning layer and the aqueous cell culture medium comprises an extra-cellular matrix (ECM) protein or macromolecule mimicking the cell adhesive properties of ECM proteins, and wherein the protein or macromolecule mimicking the cell adhesive properties of ECM proteins comprises a cell adhesive peptide.

23. The method of claim 22 wherein the cell adhesive peptide sequence comprises a peptide sequence selected from the group consisting of RGD, YIGSR, IKVAV, GFOGER and PHSRN.

24. The method of claim 1, further comprising incubation in a protease such as thrombin after assembly of the self-surface-activating amphiphilic protein or self-surface-activating amphiphilic peptide at the liquid-liquid interface.

25. Use of a culture system comprising:
a. an aqueous cell culture medium; and
b. an oil or lipid phase, wherein the oil/lipid phase is functionalised with a conditioning layer that is disposed between the aqueous cell culture medium and the oil/lipid phase and the conditioning layer comprises a self-surface-activating amphiphilic protein or self-surface-activating amphiphilic peptide layer;
for the culture of adherent cells at a liquid-liquid interface.
